(19) United States
(12) Patent Application Publication
(10) Pub. No.: US 2011/0276109 A1
(43) Pub. Date: Nov. 10, 2011

(54) Apparatus, system and method for music enjoyment and ability testing and rehabilitation retaining with cochlear implants

(76) Inventor: Lee R. Bartel, Toronto (CA)

(21) Appl. No.: 12/917,807

(22) Filed: Nov. 2, 2010

Related U.S. Application Data
(60) Provisional application No. 61/257,386, filed on Nov. 2, 2009.

(51) Int. Cl. A61F 11/04

(52) U.S. Cl. 607/57

(57) ABSTRACT

The apparatus, system and method of the invention provides a tool to measure, diagnose and retrain the ability to enjoy sound and particularly musical sound for post-lingually deafened cochlear implant recipients. The invention consists of computer driven audio and visual stimulus presentation apparatus, selected and stored musical scores, subject response apparatus, and means to access the cochlear implant which act in conjunction with fundamental components, namely:

(1) Firstly, a Sound Enjoyment Assessment Tool (SEAT),

(2) Secondly, a Perceptual Discrimination Assessment Tool (PDAT) consisting of 2 parts,

1. a pitch discrimination assessment (PA) tool, and

2. a timbre discrimination assessment (TA) tool,

(3) Thirdly, a Music Perception Ability Tool (MPAT) which functions both as an assessment and as a retraining tool, and

(4) Fourthly, a Timbre Perception Ability Retraining Tool (TPART).

The initial components SEAT, PDAT, and MPAT are used together principally as a composite Diagnostic Tool. When used “once through” SEAT produces a set of diagnostic scores of the patient's enjoyment of music, PDAT produces diagnostic scores of pitch and timbre discrimination ability, and MPAT produces a profile of scores related to the person's perceptual abilities related to music patterns and melodies. Repeated uses can be part of a rehabilitation program alone or in conjunction with the remainder of the invention.

The later components MPAT and TPART are additionally used as individual and composite Retraining Tools functioning as mastery-based, multi-level training systems premised on subject/system interaction in practice followed by increasing requirements for attention and complexity.

This application is related to and claims priority from U.S. Provisional Patent application No. 61/257,386 filed Nov. 2, 2009. Application No. 61/257,386 is hereby incorporated by reference.

### BACKGROUND

1. Field of the Invention

The invention relates to the application of functional musical and sound technology to the diagnostic and rehabilitation requirements of deafened persons, particularly those who are profoundly deafened and wearing a cochlear Implant.

2. Description of the Prior Art

In most cases the CI is implanted in the patient and is electronically connected to an exterior processing unit worn behind the ear in the immediate vicinity of the CI. The processing unit includes a sound receiver, analog and digital processors, amplifiers and electronic outputs to the cochlear implant. The implant further processes the electronic signals and delivers stimulating electronic outputs directly to the patient's cochlea. The most recent improvements include an available direct connection between external processing apparatus and the processing unit as by a audio cable or USB connection.

Perceptual difficulty in respect of music is related to rhythmic, tonal, and textural complexity levels. For CI recipients rhythmic processing is least impaired.

Additionally, the attention demand level for individuals is related to music complexity levels.

Much of the prior art has been directed to increasing the accuracy and density of the signals delivered by the CI device in the best place for processing by the user. This is in an effort to provide perfection of the input's representation of the actual ambient sounds.

A significant variable in a cochlear implant recipient's potential to enjoy music is the ability to sustain attention and to employ selective, alternating, and divided attention in the context of musical perceptual processing.

Most existing music in most styles involves greater complexity that is readily processed by CI users.

### Prior Art

Some advances in brain testing and rehabilitation are reported in the prior art listed below (not in order):

1. 2002 0130 Music Attention Training Program—ONF final report Jan. 31, 2002
2. 2006 0626 Music Attention Training Program—P MT16Rogerclean re: brain damage
3. 2002 0305 U.S. Pat. No. 6,352,345 to ZOLTEN on rehabilitation of attention,
4. 2002 0820 U.S. Pat. No. 6,435,878 to REYNOLDS
5. 2005 0405 U.S. Pat. No. 6,875,181 to KEJIMOTO

### 6. 2008 0214 USPPA 2008/0039902 to HARTLEY

7. 2009 0903 USPPA 2009/0222064 to FALTYS re programmable implant
8. 2009 0804 U.S. Pat. No. 7,571,005 to SEGEL
9. 1997 0506 U.S. Pat. No. 5,626,629 to FALTYS
10. 2000 1205 U.S. Pat. No. 6,157,861 to FALTYS
11. 2001 1023 U.S. Pat. No. 6,308,102 to SIERACKI
12. 1998 0929 U.S. Pat. No. 5,813,862 to MERZENICH
13. 1999 0209 U.S. Pat. No. 5,868 683 to PROTOPAPAS

### 14. 2002 0321 USPPA 2002/0034717 to JENKINS

### 15. 2002 0627 USPPA 2002/0078750 to WRIGHT

### SUMMARY OF THE INVENTION

Music for “practice” listening and potential enjoyment must be very carefully selected or probably contrived. Suitable musical complexity does not occur naturally in a taxonomic difficulty level form.

Since accuracy of pitch perception in relation to pre-deafened experience or normal hearing standards is impaired in CI recipients (to presented fixed hertz relationships, e.g., A3=220 hertz and A4=440 hertz), the internally referenced discrimination of the invention (e.g., what is actually heard as same or different) operating in conjunction with the implant allows greater baseline success plus an adequate starting point for rehabiliation and progressive development. Easing and grading attention demand allows for greater success potential and progressive recovery.

Music is cognitively processed in pattern “meaning units” rather than as individual pitches, and, since these are contour based and tonally relative, CI recipient internal referencing in conjunction with the CI and the invention can provide additional musical meaning.

According to the invention a significant part of recovery of musical enjoyment ability in CI users is achieved through brain rehabilitation. The sound message is provided by the invention directly to the implanted CI (direct connect) or by means of sound production in a sound attenuated space and thence is received from the implant. In prior art methods audiological perfection is sought between ambient sounds and the sound signals output from the CI device into the brain.

Systematic rehabilitation employing the invention's graded complexity levels of musical sounds in relation to attention demand levels is effective at restoring enough musical perception to improve enjoyment of music and, thereby, the utility and enjoyment of other ambient noises. Musical materials and methods enhance the rehabilitation of attention and memory deficits.

Graded complexity levels provide for increasing contour perception, namely rising and/or falling patterns. Pitch patterns are provided in contours and the subjects pick out a particular pattern from the set. The invention starts with patterns that are composed of elements with a substantial frequency difference and then proceeds to narrower and narrower differences. This focuses both testing and rehabilitation on progressively more refined pitch differences.

The internally referenced hedonic listening of the invention more likely results in “enjoyment” than critical comparative listening techniques of the prior art. Since perceptual difficulty taxonomy in music does not occur naturally it is provided by the graded application of the invention.

Repeated usage of the multi-stage testing process of the invention improves subject implant wearer's scores on tests that are good measures of brain function recovery more than those subjects who did not receive rehabilitation training from the invention.

Preferably, the invention provides visual guidance as an adjunct to auditory perception and aural memory and success feedback as a motivational factor.

The invention includes either a direct connection between the test and rehabilitation apparatus and the cochlear implant device or a sound attenuated space for subject interaction and means to deliver both musical sounds and visual images to the test subject through the implanted CI, such as audio equipment and video screens. Means are provided for the subject to respond to either or both of the musical sounds and/or the visual images perceived as a result of the CI processing. Responses are timed and graded. Preferably, means are provided for practice sessions at each level. Assessment and performance data are collected and processed before going on with assessment and/or rehabilitation. Musical sounds and visual images and the respective timing of assessment and/or rehabilitation steps are presented based upon advancing musical complexity and the subjects performance. The extent of repetition are based upon scoring improvements made in real time and/or subsequently processed.

### DESCRIPTION OF THE FIGURES

FIG. 1 is a schematic drawing of the Perceptual Retraining System of the Invention in respect of the Pitch Pattern section. The basic system functions in this fashion through 6 levels of difficulty in the preferred embodiment.

FIG. 2 is a schematic drawing as in FIG. 1 in respect of the Patterns in Melodies section. The basic system functions in this fashion through multiple melodies.

FIGS. 3 and 4 are tables showing the test rating methodology of the preferred embodiment of the invention for the preferred SEAT, Music Enjoyment Assessment, and Pitch Discrimination Scoring in FIG. 3 and Timbre Discrimination Scoring and Pattern and Melody Perception Scoring in FIG. 4, respectively.

FIGS. 5 through 61 inclusive show the individual steps in carrying out the the test and rehabilitation system in interacting with the subject by the sounds, visual symbols and guidelines generated by the preferred embodiment of the invention.

FIGS. 62 through 70 inclusive are tables showing test results gathered from noted subjects in carrying out a pilot version of the invention as described.

FIG. 71 is a schematic diagram of alternative preferred embodiments of the invention A and B.

### DESCRIPTION OF THE INVENTION

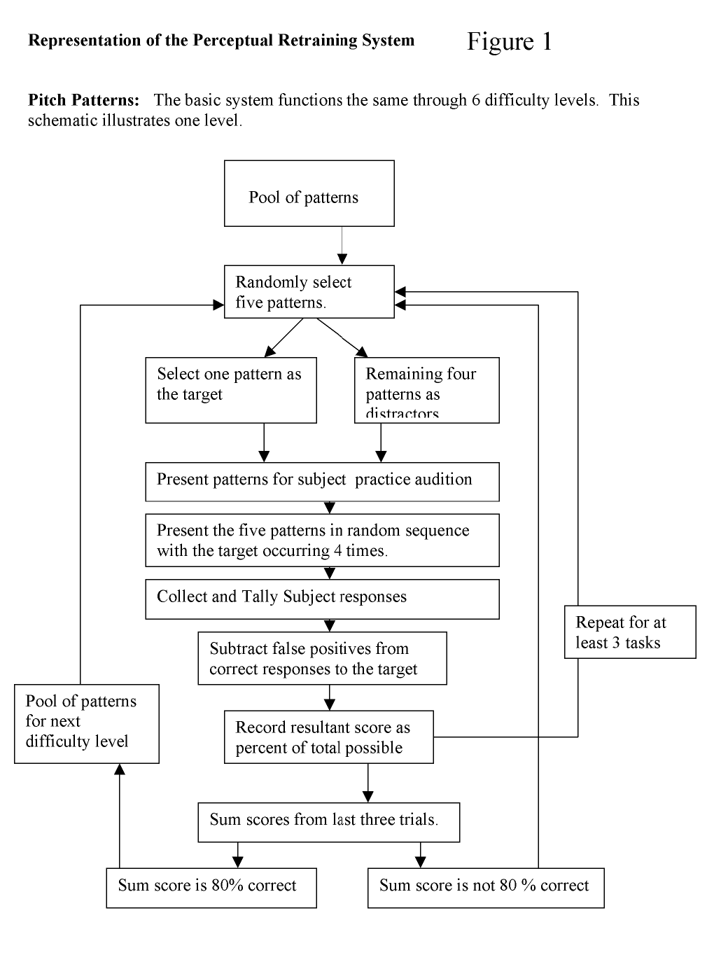

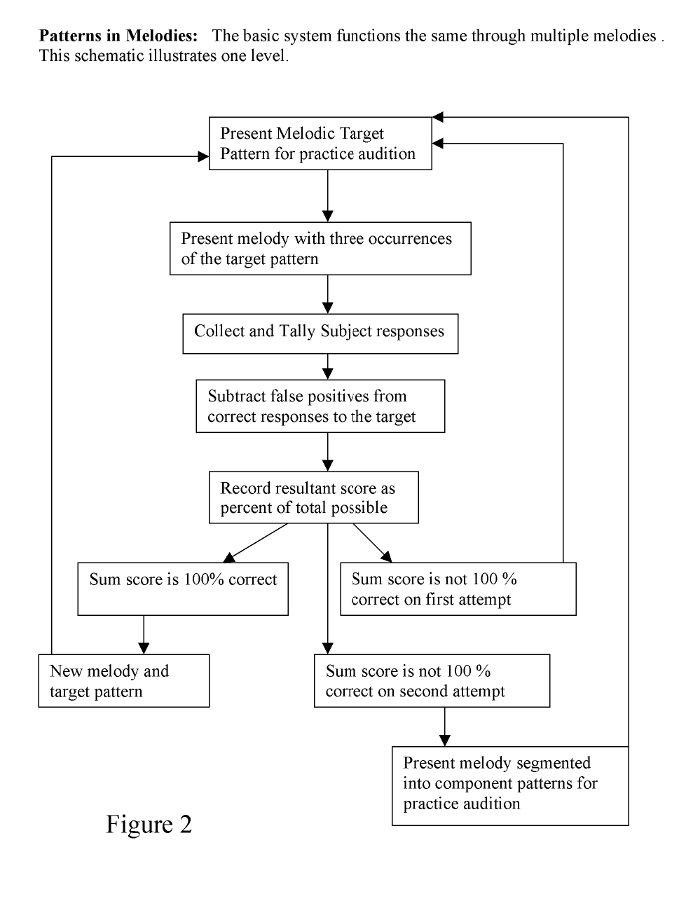

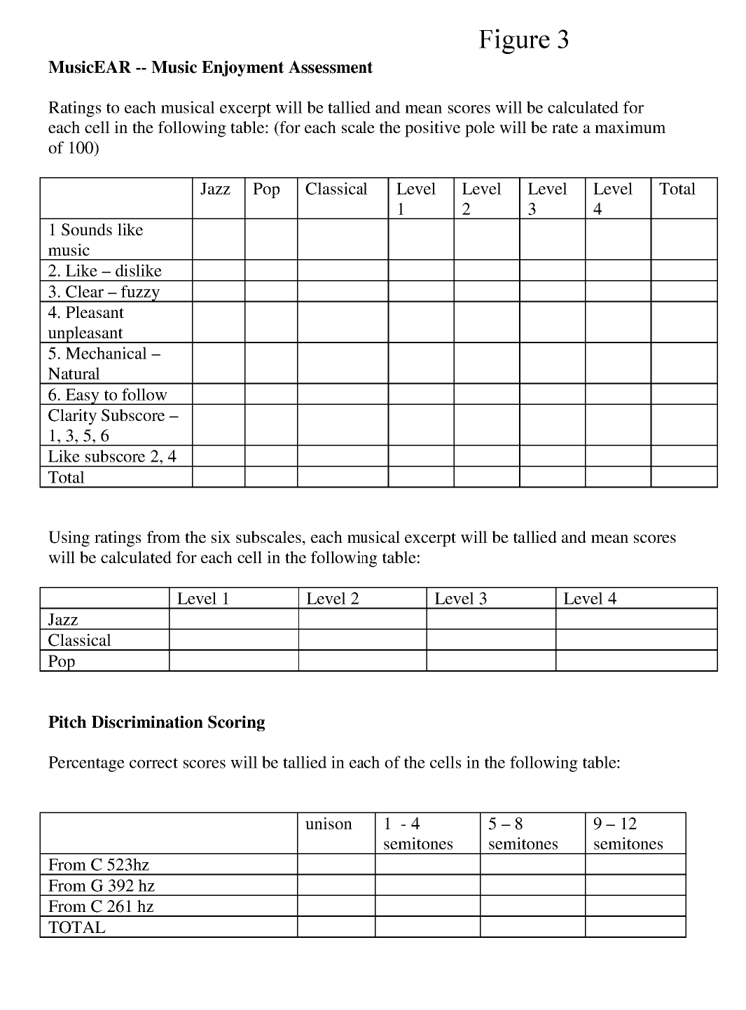

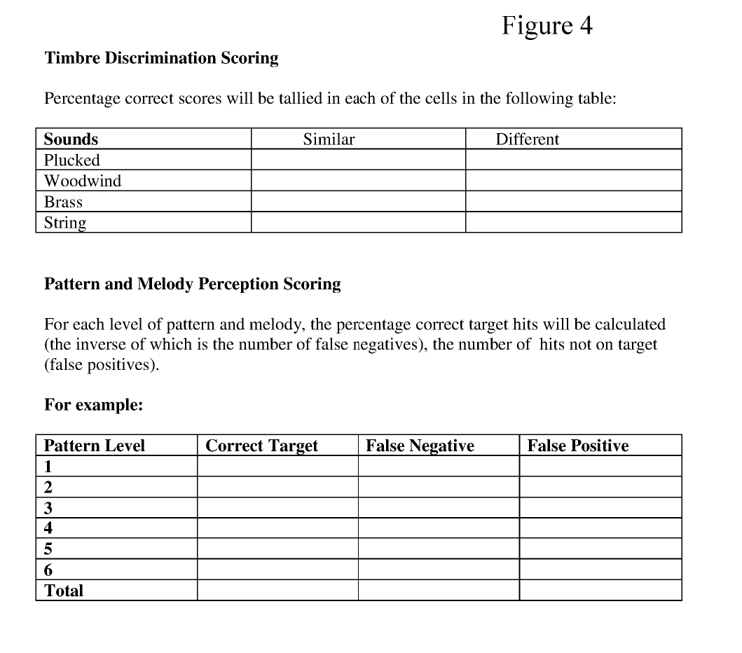

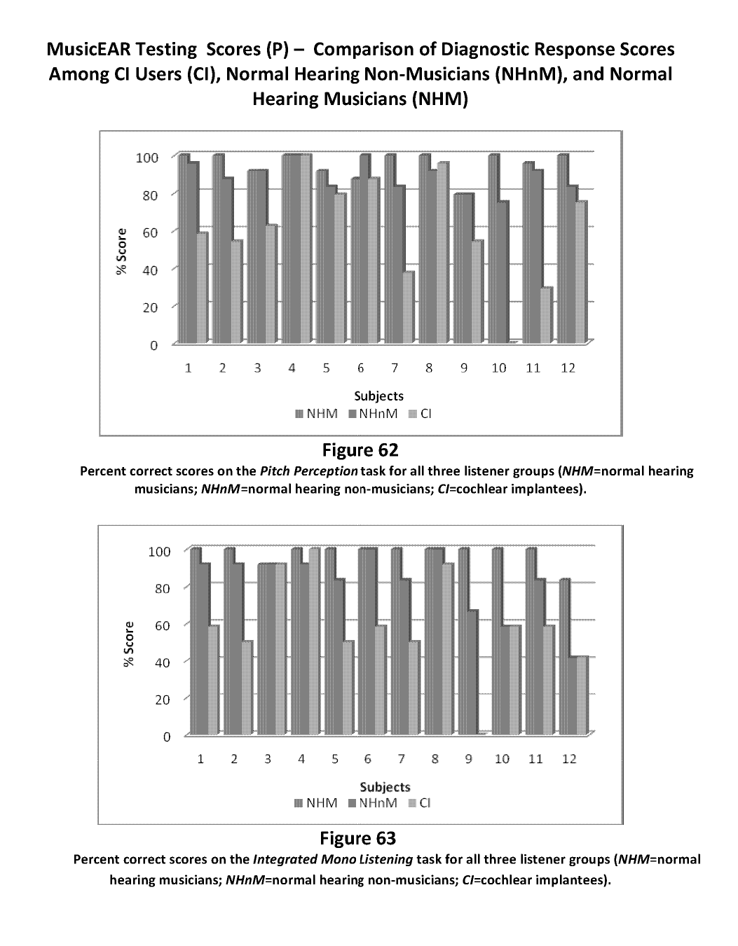

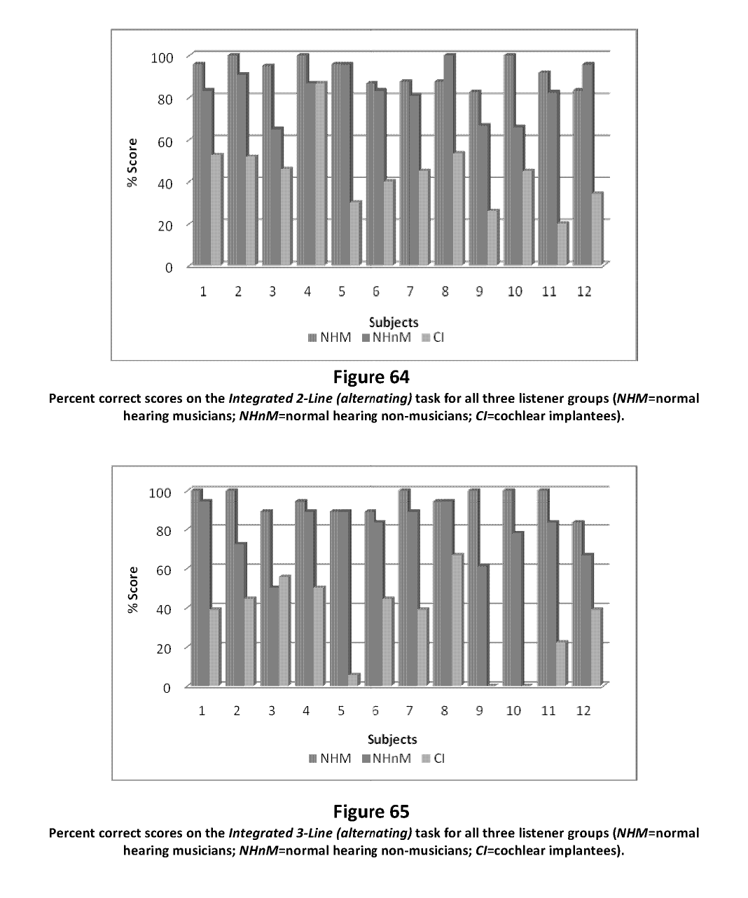

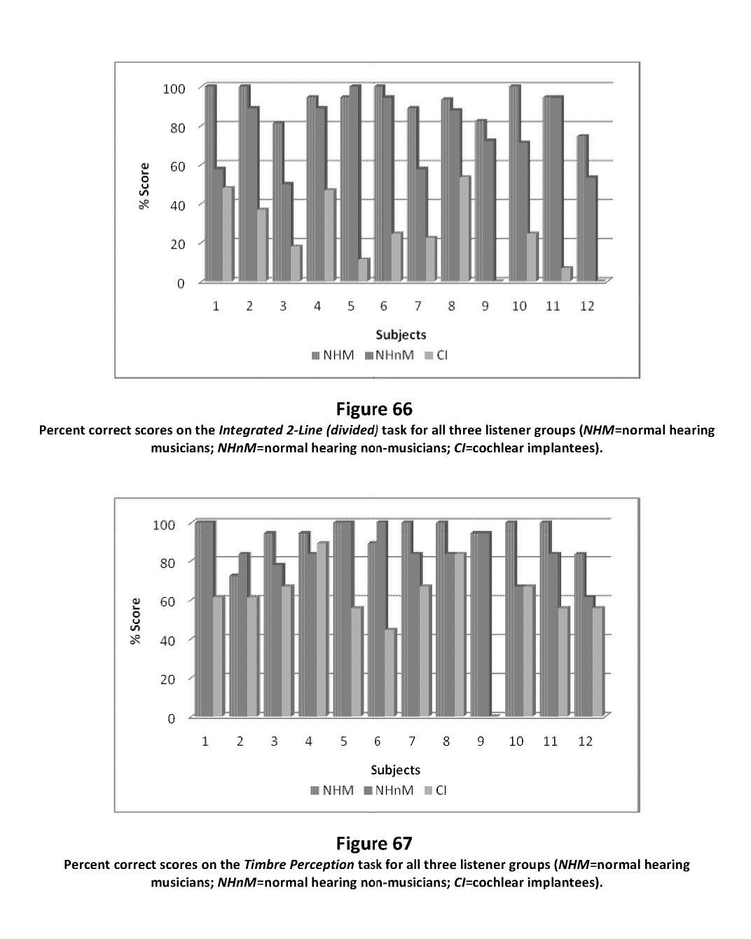

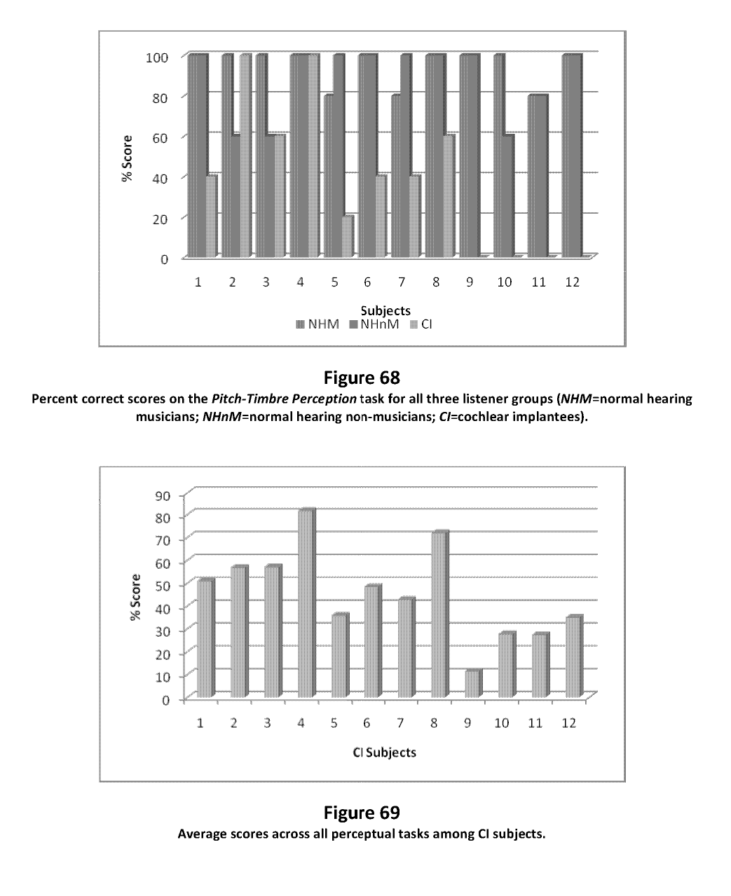

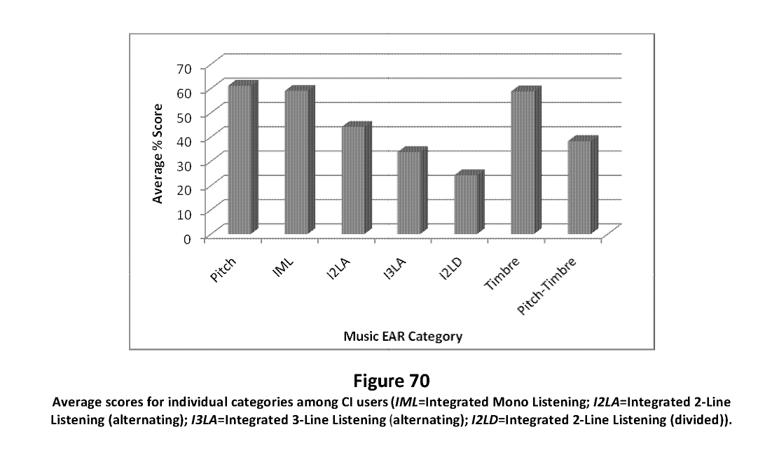

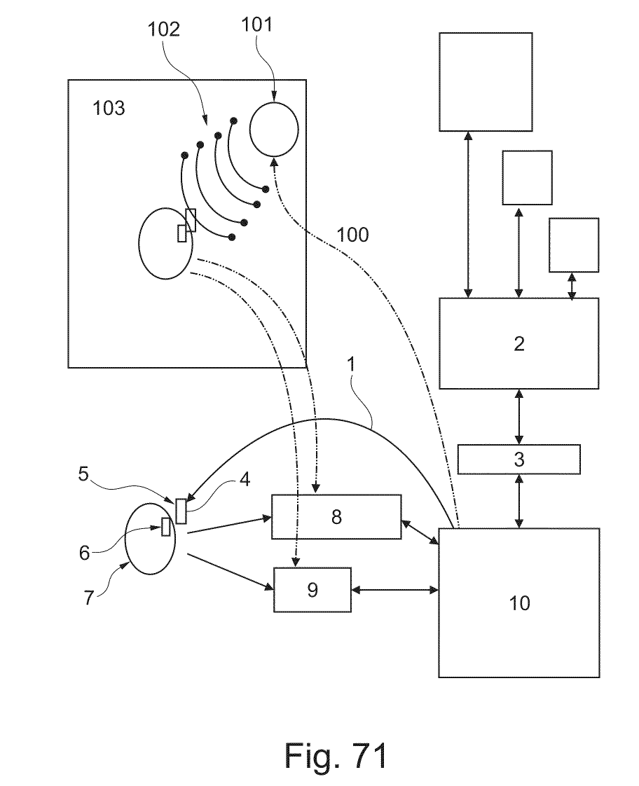

The preferred embodiment of the processing components of the system, procedure and method of the invention is shown in FIGS. 1 through 61 inclusive. Pilot test results are shown in FIGS. 62 through 70. A schematic of the computer driven audio and visual stimulus presentation apparatus, computer driven processing apparatus, stored musical scores, subject response apparatus, and means to access the cochlear implant which act in conjunction with the 4 fundamental components is shown in FIG. 71 in alternative forms A (direct connect) and B (attenuated sound space). In the preferred embodiments of FIG. 71, the directly connected preferred embodiment, alternative A, includes digital audio signal output by data cable 1 from the CPU device 2 (either locally or at a distance as by the internet as at 3) and to data cable 1 connected directly to the USB external connection 4 of the exterior processing unit 5 of the implant 6.

The implant 6 sends its signals to the patient or recipient 7 who responds to choices by keyboard 8 or input device 9. Visual presentation screen 10 provides stimulus to the patient 7.

In alternative B, a data cable 100 provides sound singals to speaker 101 which then broadcasts the selected sounds 102 in attenuated space 103 to the 160 implant external processing unit 5. The patient responds as in alternative A. Preferably, the first component (SEAT) of the invention provides a Music Enjoyment Assessment Tool including 2 concepts interacting with cochlear implant recipients' response to music: namely musical style and textural complexity.

For each style a preferably related texture level is selected and presented to the subject. Diagrammatically this is shown in Table A for each of ‘single line’, ‘2-part’, ‘multi-part’ and ‘complex’ textures of the music or sound.

TABLE A

Texture Level

| Style | Single line | Two part | Multi-part | Complex |
|---|---|---|---|---|
| Classical | | | | |
| Popular | | | | |
| Jazz | | | | |

Twelve musical excerpts, for example, taken from Table B below for each level are provided as typical conservative exemplars of the style. Simple textures are essentially monophonic or with minimal accompanying lines. Two-part selections are basically single melodic lines with sparse accompaniment. Multi-part selections use several instruments but with linear clarity, little vertical density, and a clear melodic component. Complex textures have more than two lines with considerable “voice” interaction. Excerpts are about 30 seconds in length and are presented in random order.

TABLE B
Music Materials - 30 second selections from these sources
Jazz

| | |
|---|---|
| Level 1 | Artist: Brantford Marsalis Quartet |
| | Piece: A Thousand Autumns, B. Marsalis, Steeplone Music, Inc. |
| | Album: Requiem CK69655 Columbia, Sony Music Entertainment (1999). |
| Level 2 | Artist: Ken Poplowski and Howard Alden |
| | Piece: Fading Star, Willie “The Lion” Smith, EMI Mills Music, Inc. |
| | Album: Encore. CCD4654, Concord Jazz (1995). |
| Level 3 | Artist: The Brantford Marsalis Quartet |
| | Piece: Mo' Better Blues Bill Lee |
| | Album: Music from Mo' Better Blues CK 46792, CBS Records Inc. (1990). |
| Level 4 | Artist: Rob McConnell & The Boss Brass |
| | Piece: Rockin' in Rhythm, arr. Rob McConnell |
| | Album: Don't' Get Around Much Anymore CCD 4661, Concord Jazz (1995). |

Classical

| | |
|---|---|
| Level 1 | Artist: Yo Yo Ma |
| | Piece: Simple Gifts (traditional) arr. Meyer |
| | Album: Classic Yo-Yo, SK 89667, Sony Music Entertainment, (2001) |
| Level 2 | Artist: Hungarian State Orchestra, Kerry Stratton conducting. |
| | Piece: Liszt Fantasy on Beethoven's Ruins of Athens. |
| | Album: Liszt, HCD 31525, Hungaroton Classic Ltd. (1994). |
| Level 3 | Artist: New Philharmonia Orchestra, Edo DeWaart, Conductor, |
| | Arthur Grumiaux, Violinist |
| | Piece: Bach, Violin Concerto BWV 1041-1042 |
| | Album: Bach Violin Concertos, 420-700-2 Phillips Silver Line |
| Level 4 | Artist: Boston Symphony Orchestra, Charles Munch, Conductor. |
| | Berj Zamkochian, organ. |
| | Piece: C. Saint-Saens, Symphony #3 in C Minor, Op. 78 “Organ.” Maestoso |
| | Album: Living Stereo Audiophile Sampler, BMG Music, (1993) |

Pop

| | |
|---|---|
| Level 1 | Artist: Ute Lemper |
| | Piece: The Rose, Amanda McBroom, Neue Welt Musikverlag/WB |
| | Album: Life is a Cabaret, WMK 46049 CBS Records (1987). |
| Level 2 | Artist: Neil Diamond |
| | Piece: I am . . . I said |
| | Album: The Best of Neil Diamond, MCAD 11947, MCA Records (1999). |
| Level 3 | Artist: Bread |
| | Piece: Baby I'm a want you, Gates, WEA International Inc. |
| | Album: Simple Living MDC20013 MDC Music Design & Concepts (2000) |
| Level 4 | Artist: Tom Jones & Simply Red |
| | Piece: Ain't that a lot of love, Willia Dean Parker and Homer Brooks, Rondor |
| | Music Publishing Ltd. |
| | Album: Reload. ACD1550 Gut Records Ltd. (1999). |

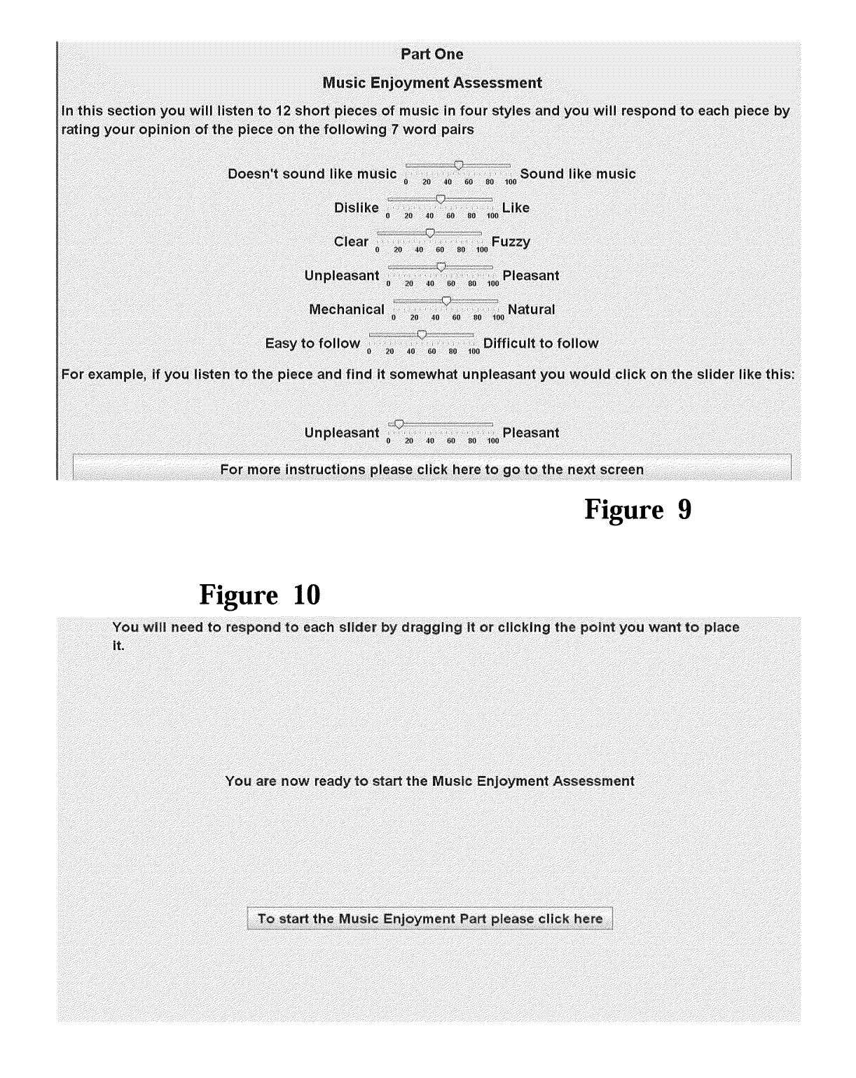

The invention requires the subject to respond as in FIG. 9 to each musical excerpt on the following 10 point rating scales as shown in FIG. 12:

| | | |
|---|---|---|
| Not enjoyable | 1 2 3 4 5 6 7 8 9 10 | Very Enjoyable |
| Doesn't sound like music | 1 2 3 4 5 6 7 8 9 10 | Sounds like music |
| Like | 10 9 8 7 6 5 4 3 2 1 | Dislike |
| Unpleasant | 1 2 3 4 5 6 7 8 9 10 | Pleasant |
| Mechanical | 1 2 3 4 5 6 7 8 9 10 | Natural |
| Clear | 10 9 8 7 6 5 4 3 2 1 | Fuzzy |
| Easy to follow | 10 9 8 7 6 5 4 3 2 1 | Difficult to follow |

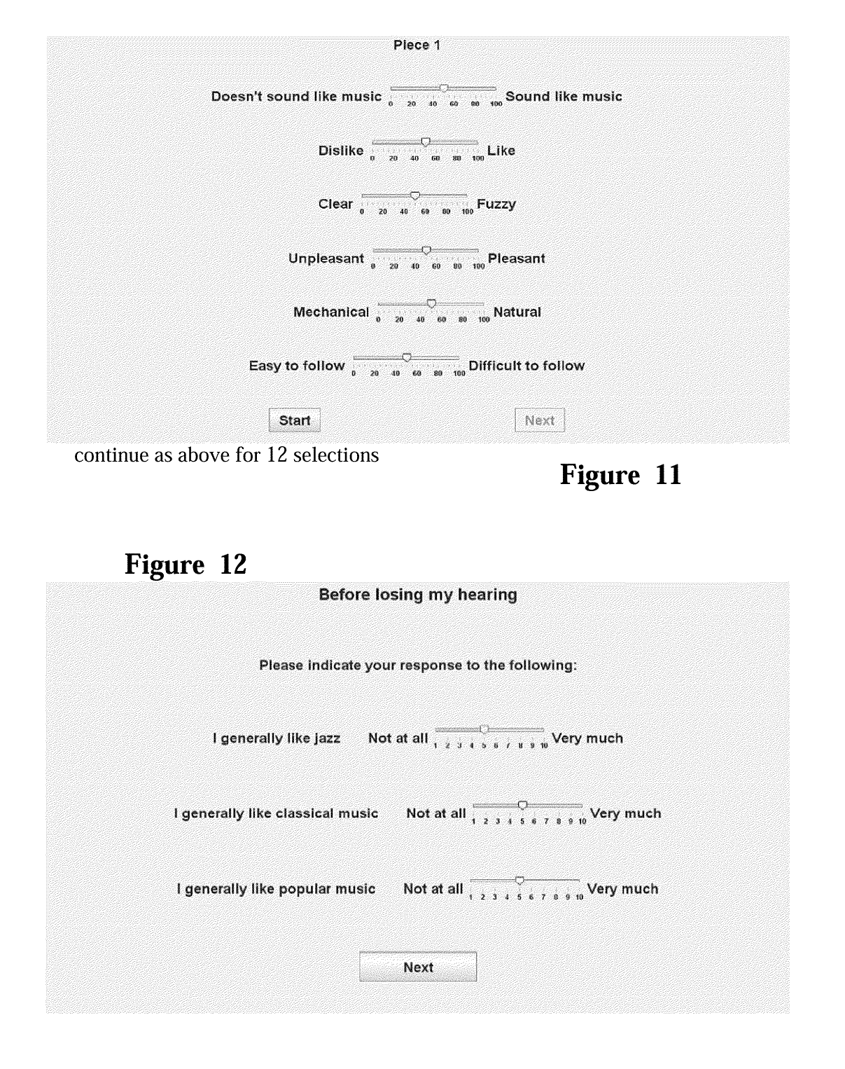

FIG. 11 shows an individual excerpt application of the steps of FIG. 9. Following the completion (FIG. 10) of the 12 musical excerpts, the subject is required to respond on the following rating scales:

1. Before I lost my hearing as shown in FIG. 12:

(a) I generally liked jazz:
Not at all 1 2 3 4 5 6 7 8 9 10 Very Much

(b) I generally liked classical music
Not at all 1 2 3 4 5 6 7 8 9 10 Very Much

(c) I generally liked popular music
Not at all 1 2 3 4 5 6 7 8 9 10 Very Much

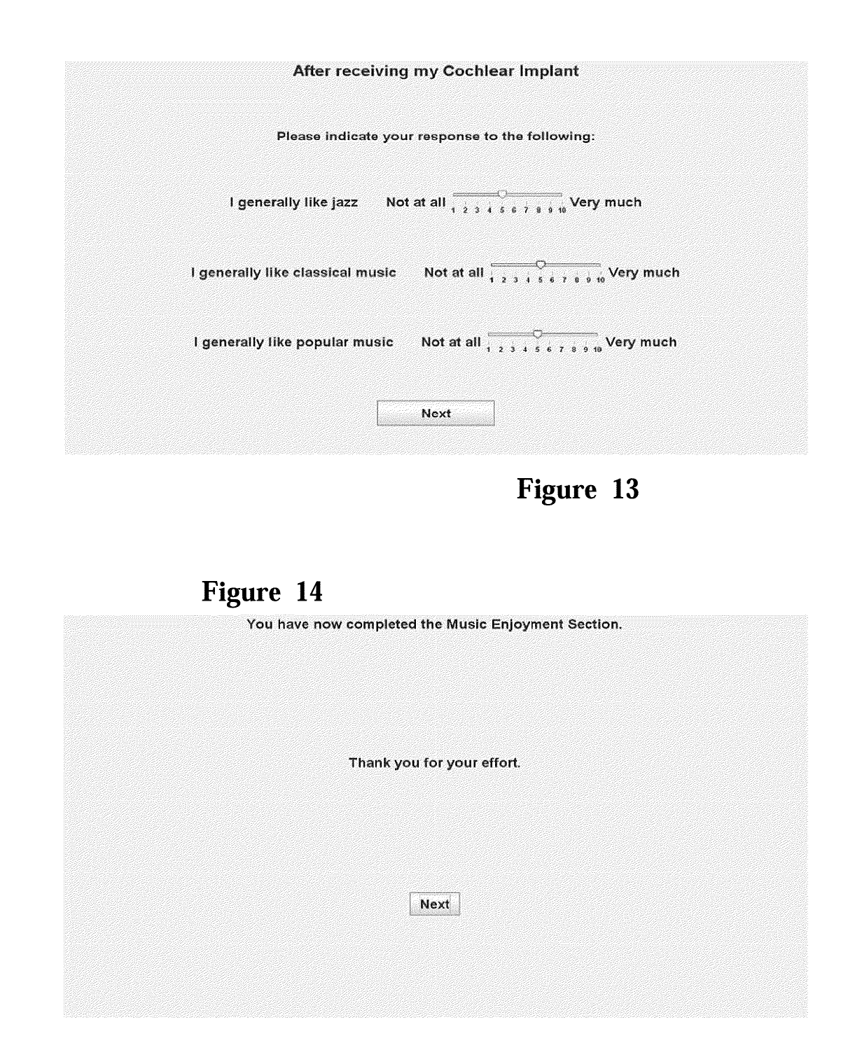

2. After receiving the cochlear implant as shown in FIG. 13:

(a) I generally like jazz:
Not at all 1 2 3 4 5 6 7 8 9 10 Very Much

(b) I generally like classical music
(c) Not at all 1 2 3 4 5 6 7 8 9 10 Very Much

(d) I generally like popular music
(e) Not at all 1 2 3 4 5 6 7 8 9 10 Very Much

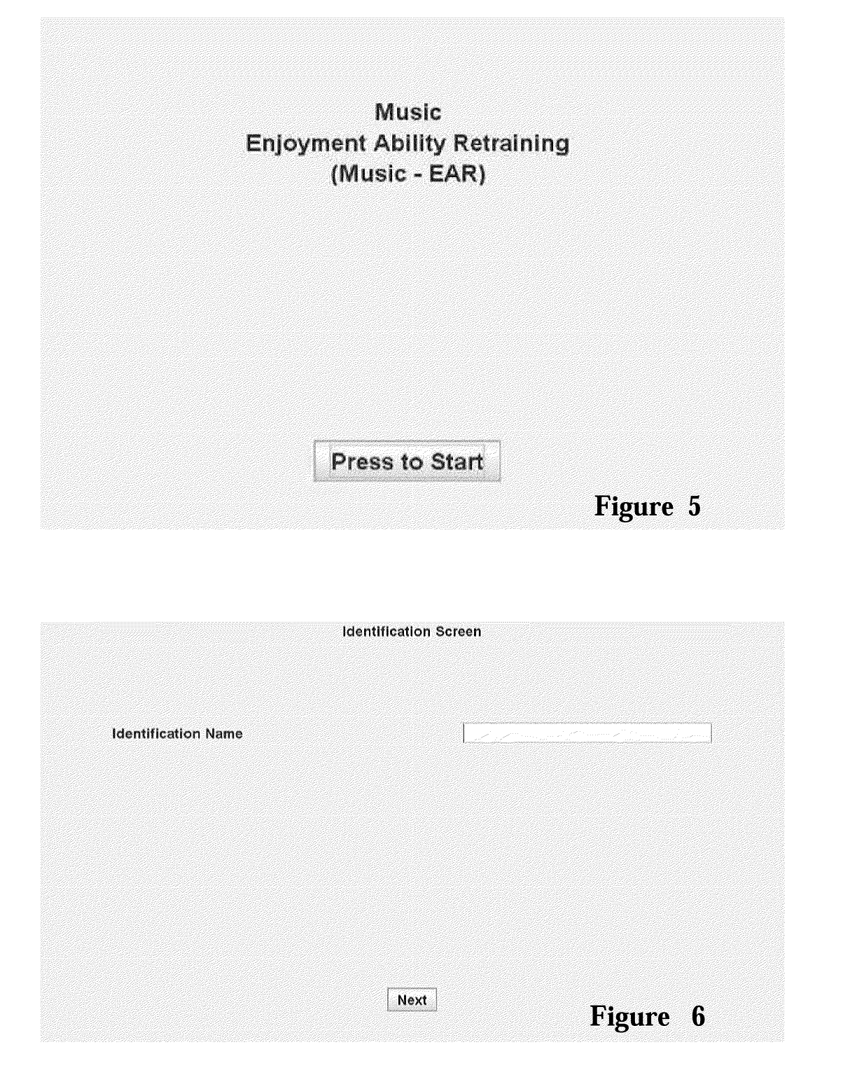

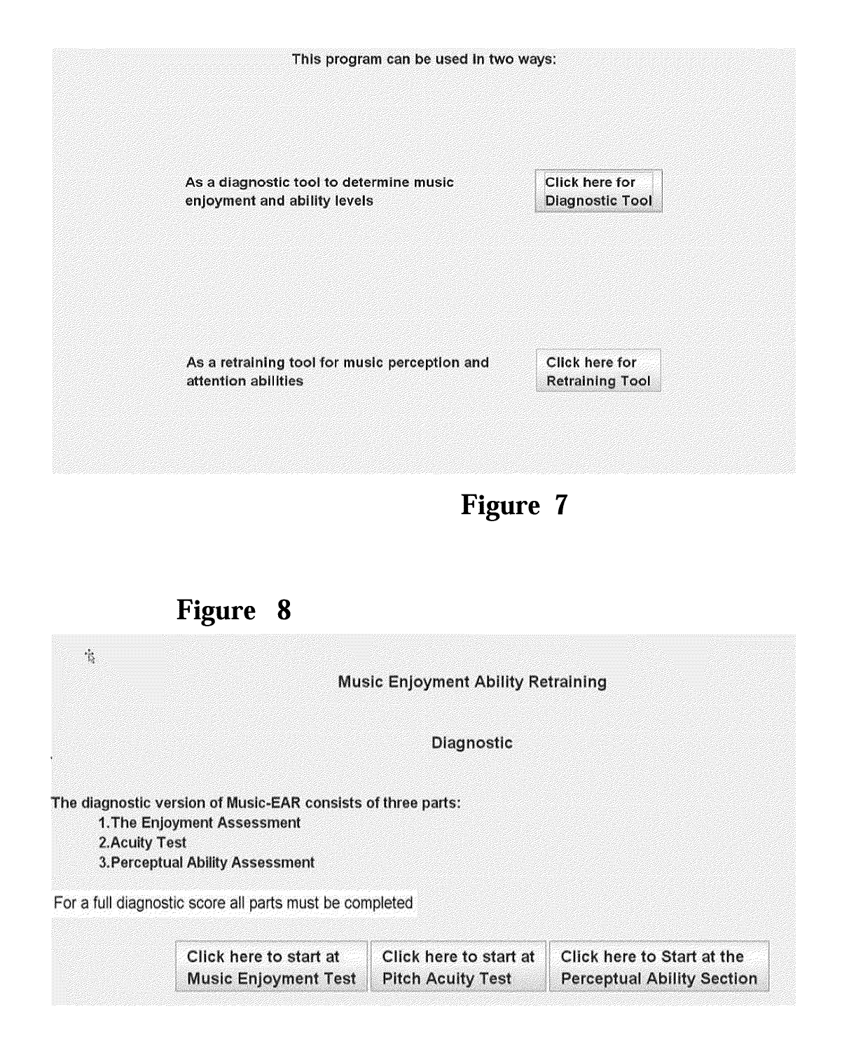

The invention provides data collection as in FIGS. 5 to 14 on the subjects reactions by style scores, texture level scores, and overall scores on each subscale. Preference scores can be used as covariants with the style subscales scores.

Preferably, the 2^{nd} Component of the invention provides a Perceptual Discrimination Assessment Tool (PDAT) which (1) identifies a target from a “line-up” of similar patterns; (2) provides graphic visual cuing for pitch relations, (3) provides visual feedback for response correctness, (4) targets response to a specific recurring motive in a melody, and (5) provides increasing perceptual complexity to correspond with attention levels, preferably comprising:

Firstly, a Pitch Discrimination Assessment Tool (PA) including 32 interval tasks presented with a practice session and individual steps as shown in FIGS. 15 to 17 to the subject for response of which:

1. 4 are unisons—the same pitch.

2. 12 are intervals of 1 semitone to 4 semitones (3 occurrences of each),

3. 8 are 5-8 semitones,

4. 8 are 9-12 semitones.

5. Of these 32 pitch pairs:

(a) 11 start on c an octave above middle c (523 hz),

(b) 11 start on second line g (392 hz),

(c) 10 start on middle c (261 hz).

Of the 28 intervals (not counting the 4 unisons), about half go down from the first pitch half go up.

The PAT pitches are presented in pairs and the subject is required to judge the second pitch higher, lower, or the same.

Scores are calculated based upon each of the four pitch difference categories and for the total.

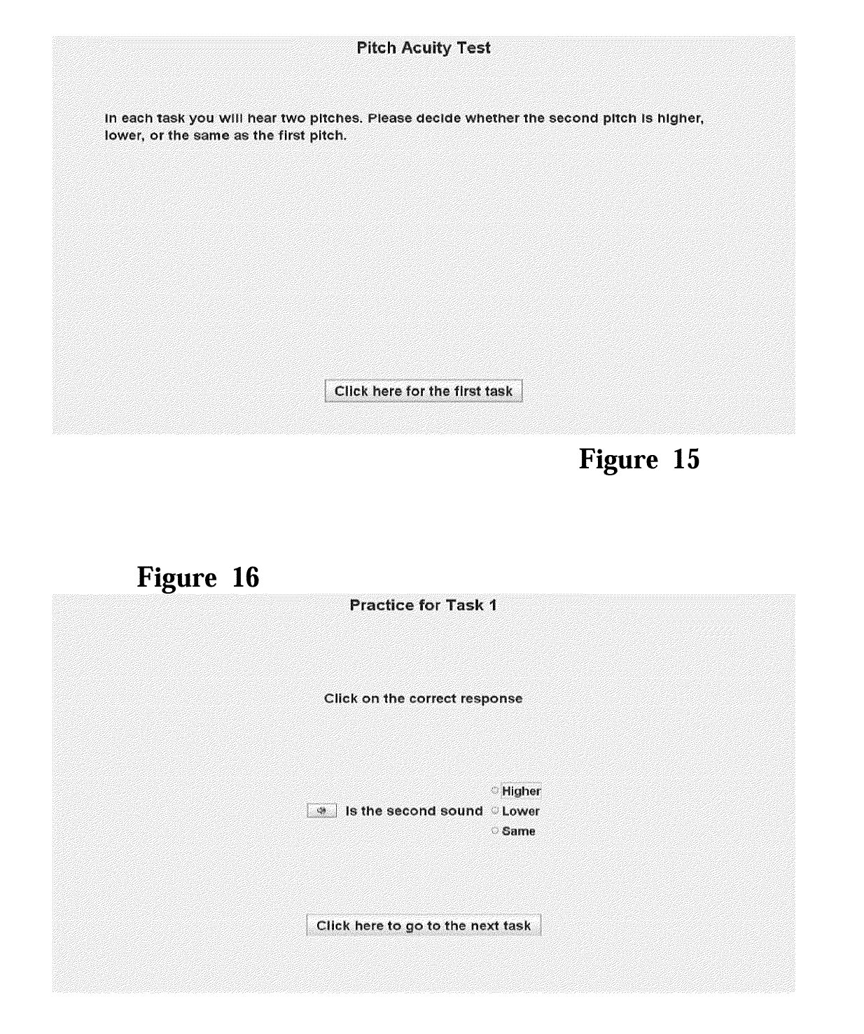

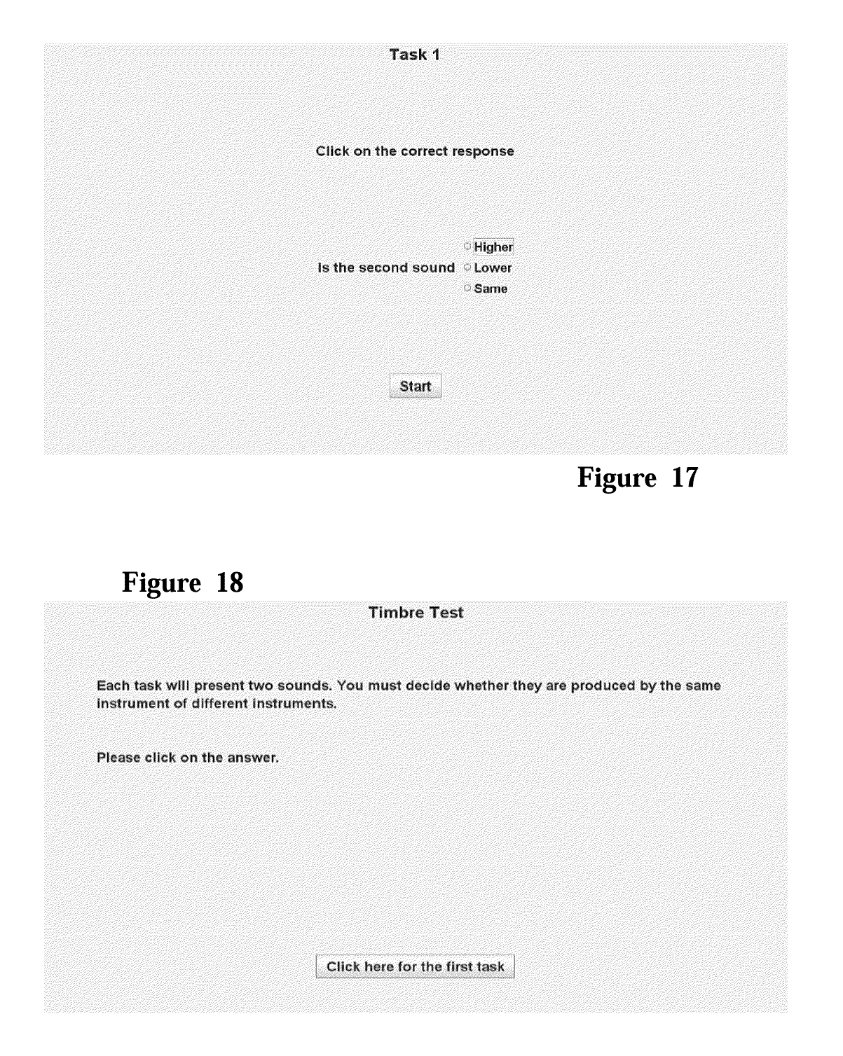

Secondly, a Timbre Discrimination Assessment Tool (TA) includes 32 timbre pairs presented to the subjects for response as shown in FIGS. 18 and 19, alternatively with a practice session as in FIG. 16. The subject is required to judge the musical sounds as “same”, “different” or “not sure” and respond.

Further preferably the timbres presented include:

1. Plucked sounds—guitar, mandolin,

2. woodwind sounds—clarinet, oboe, sax, flute

3. Brass sounds—trombone, horn

4. String—Violin

and the subject is not required to match an instrument name with a timbre pair, only to differentiate the actual timbres presented by the invention.

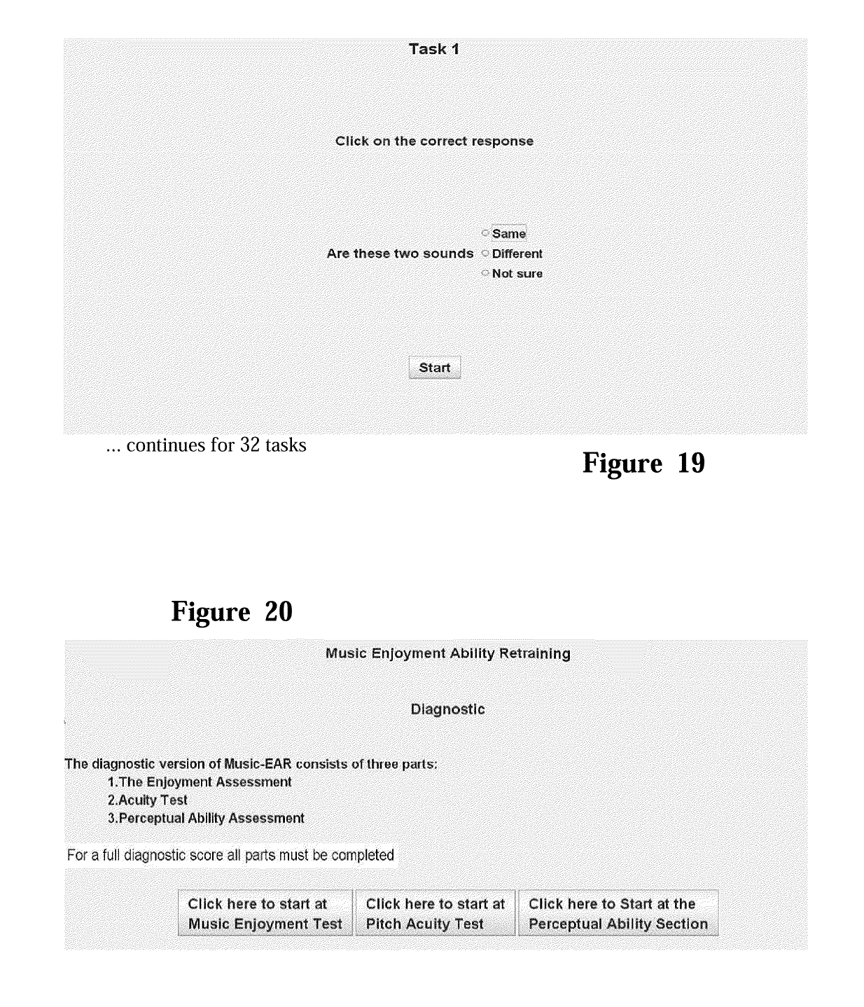

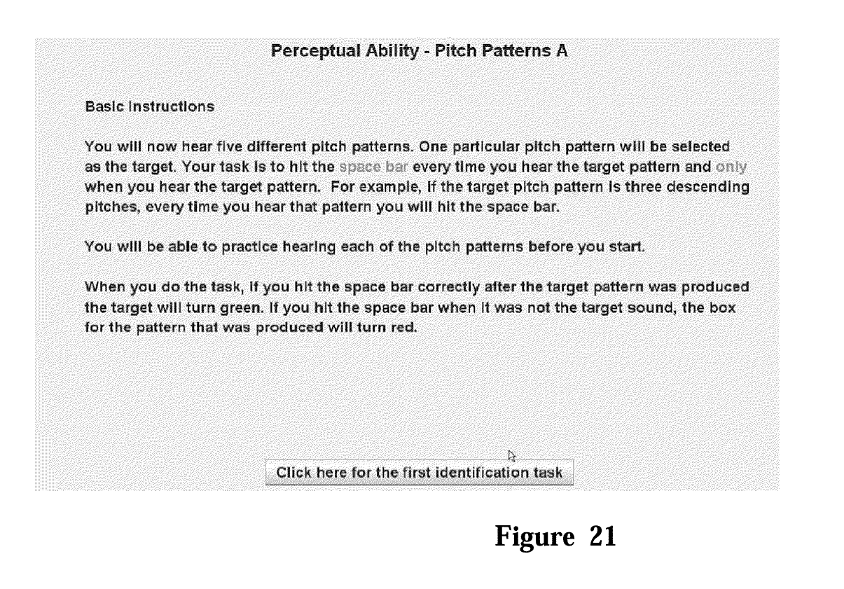

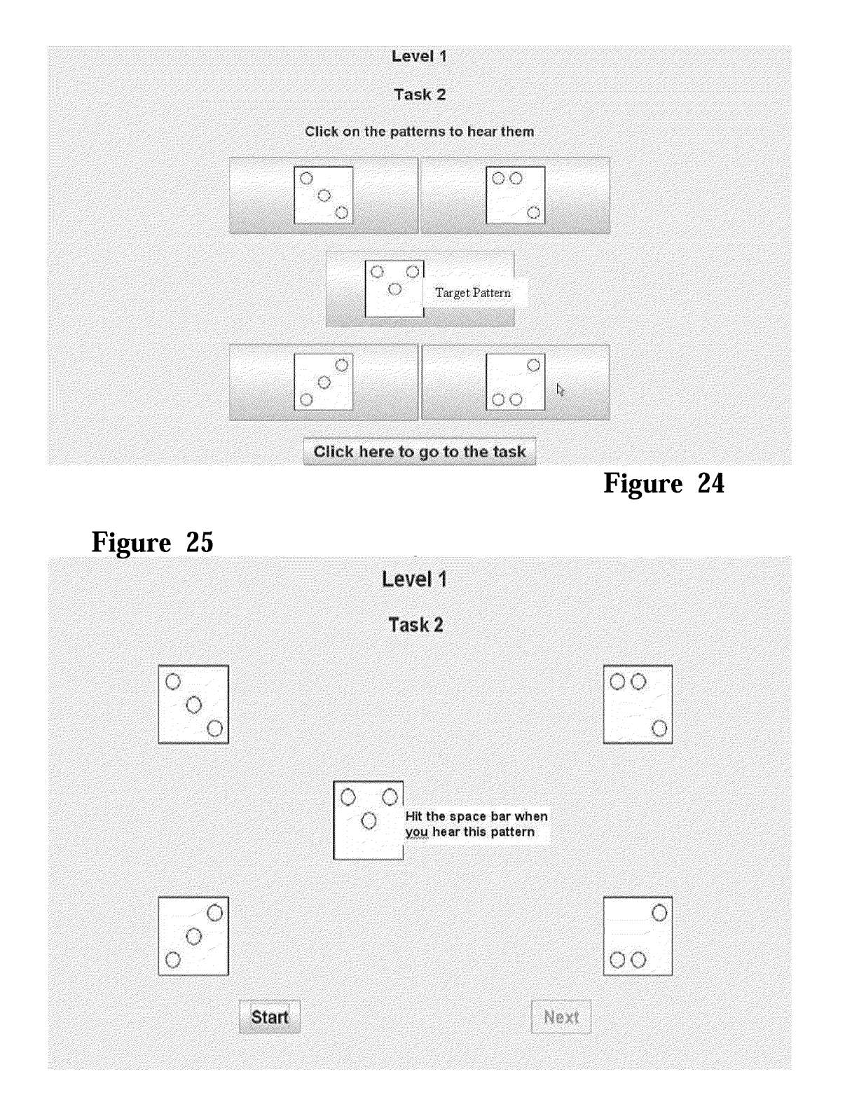

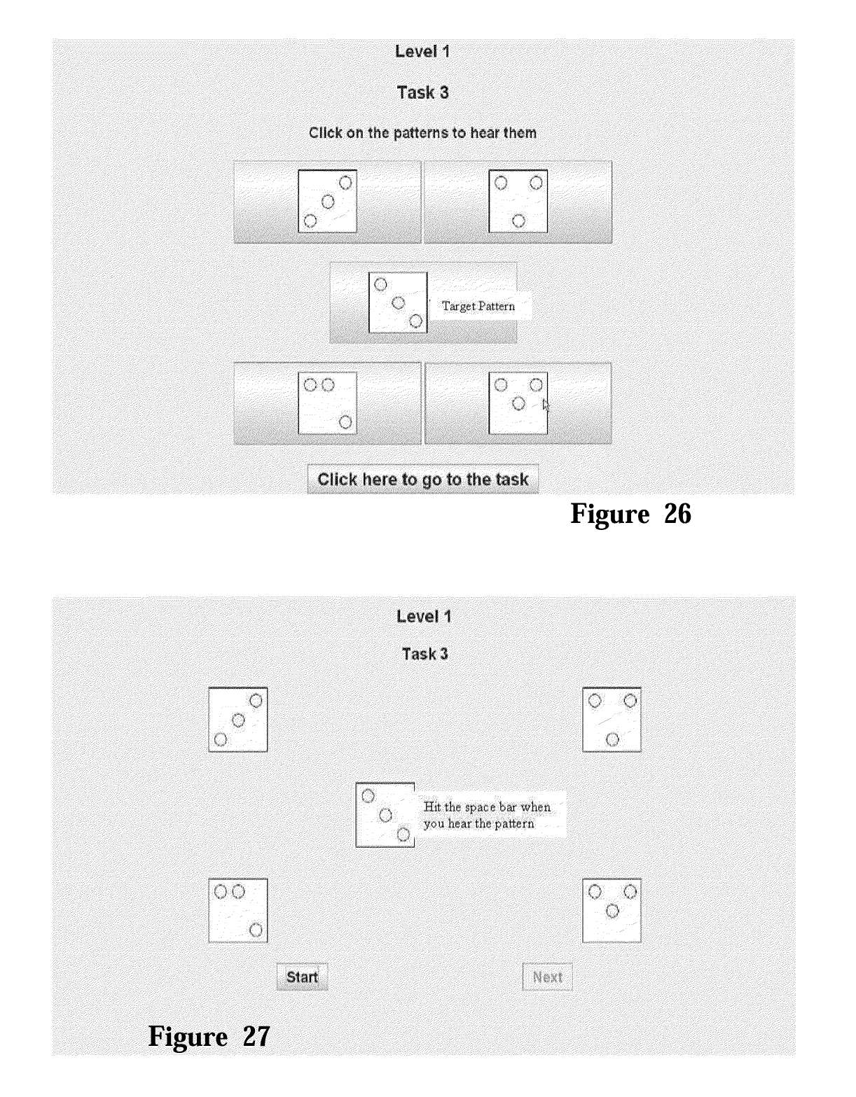

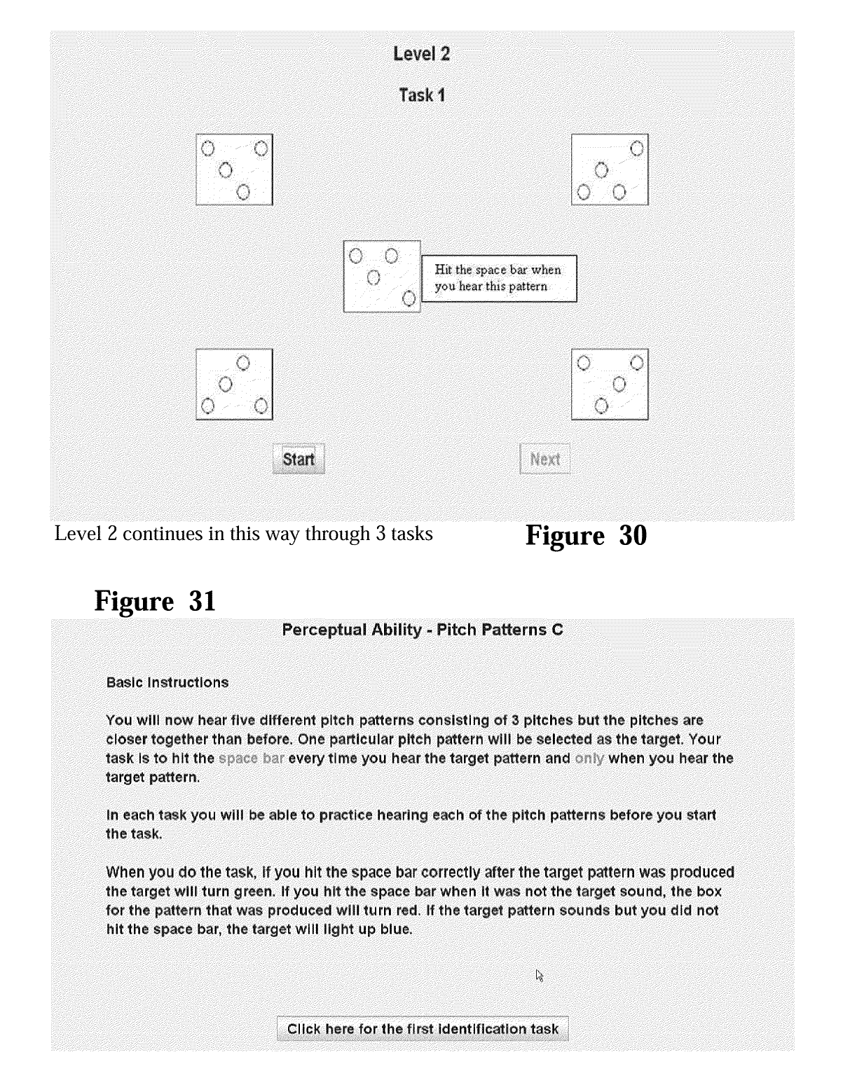

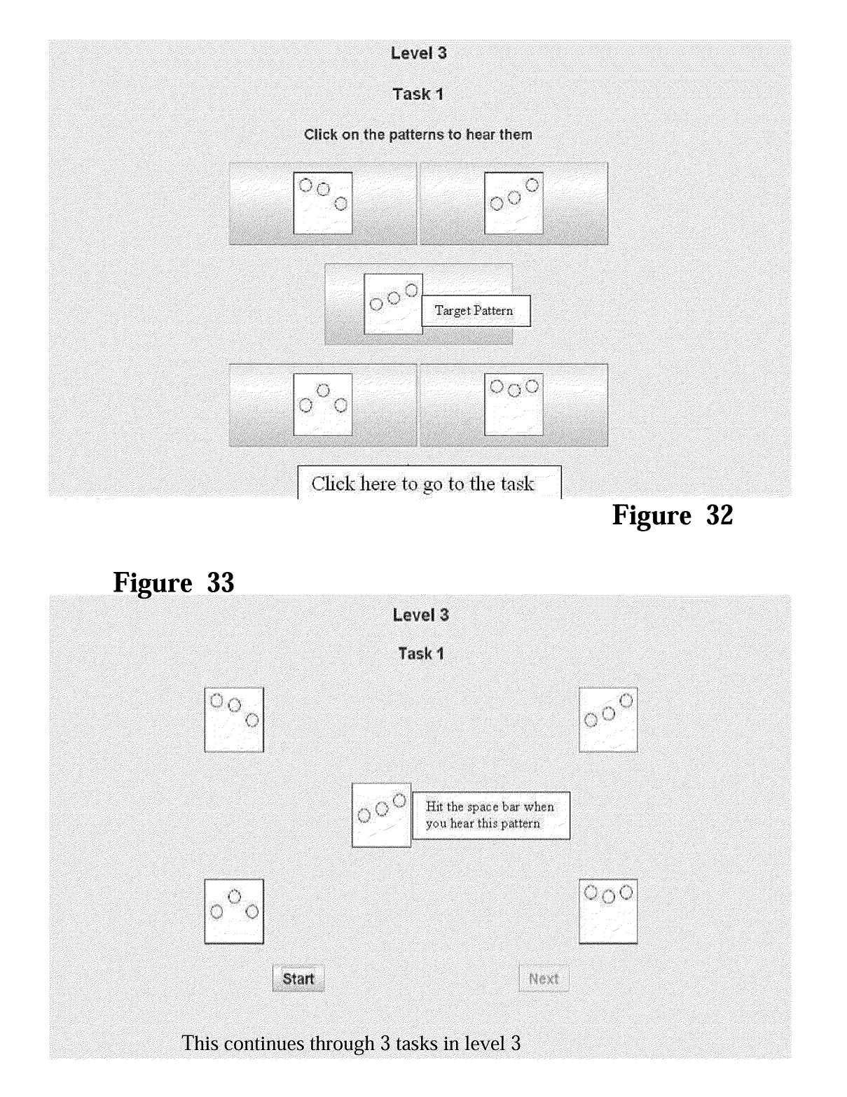

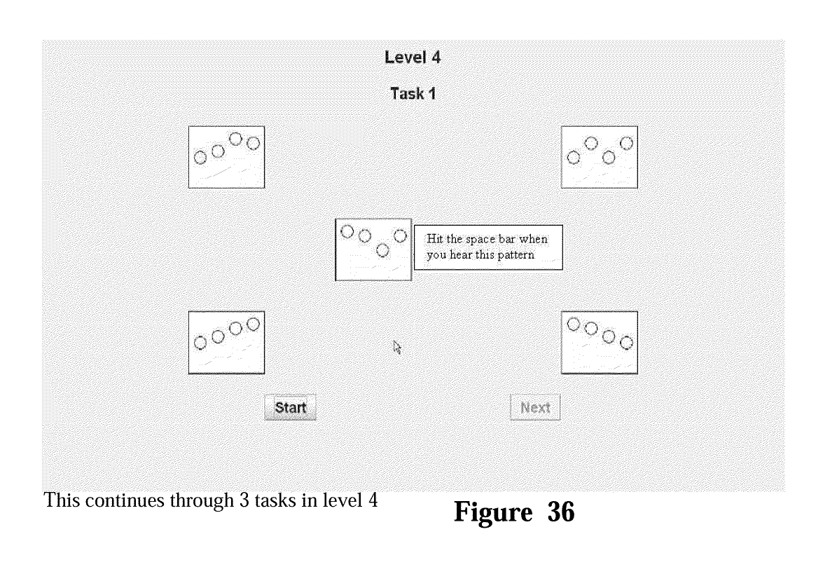

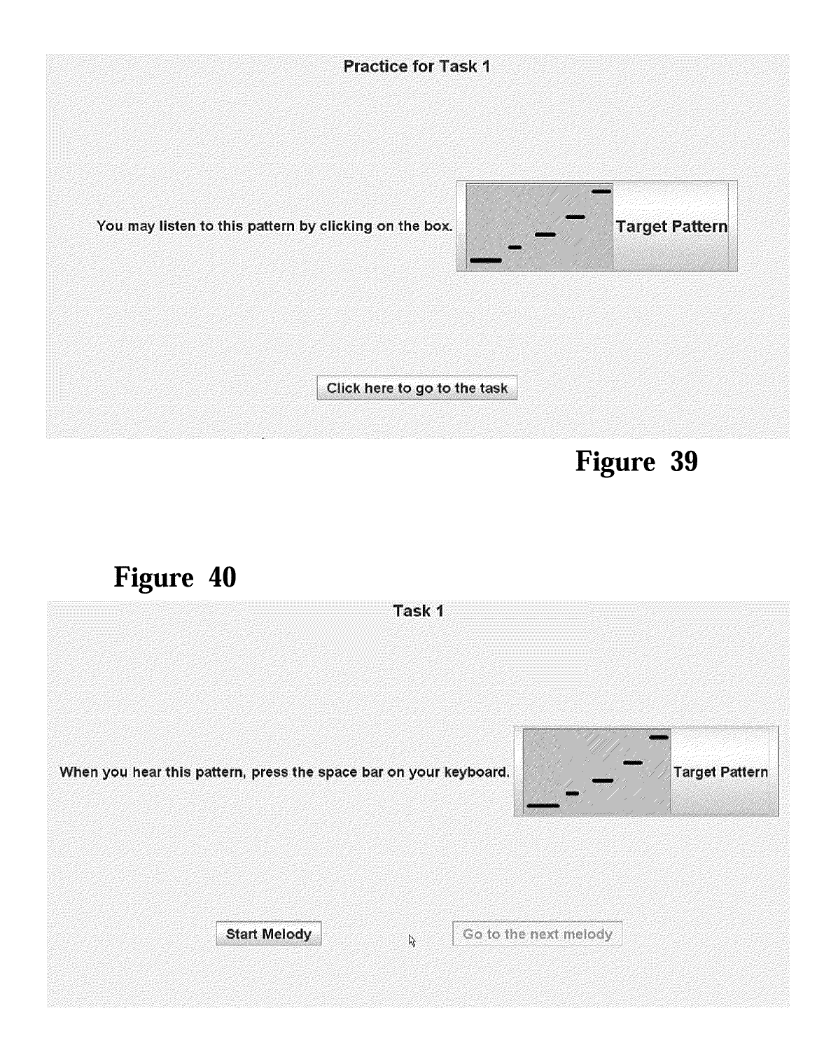

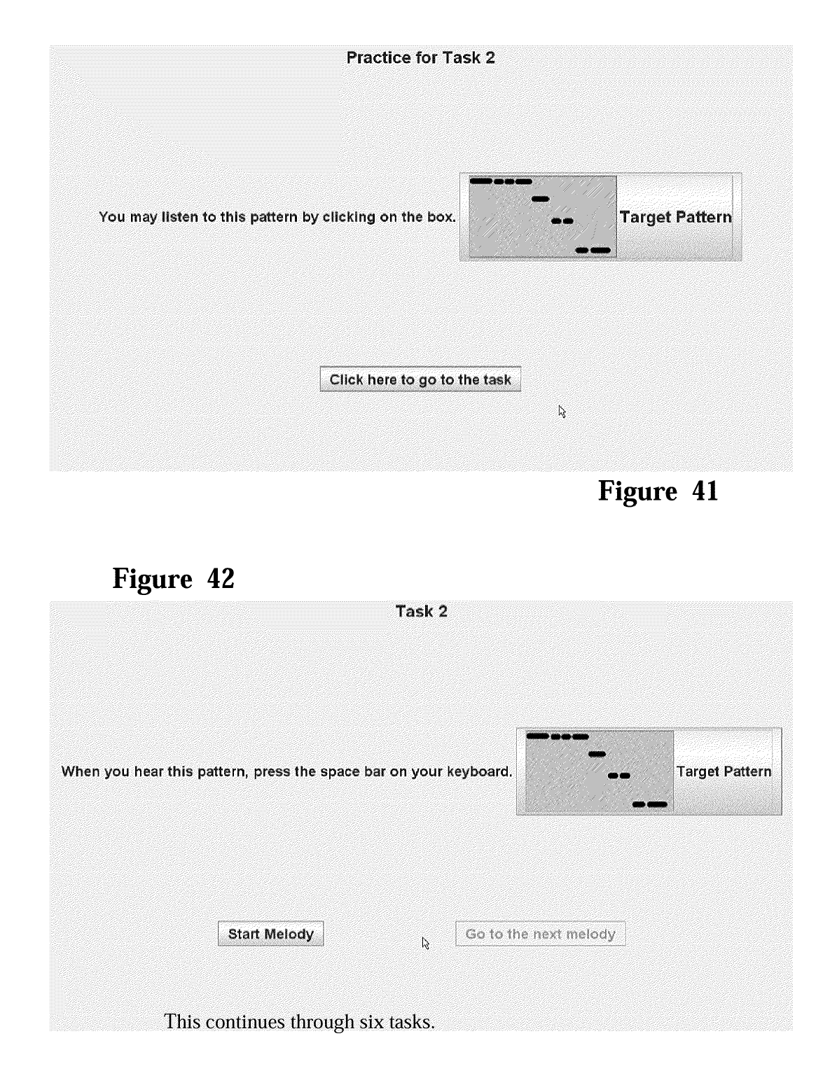

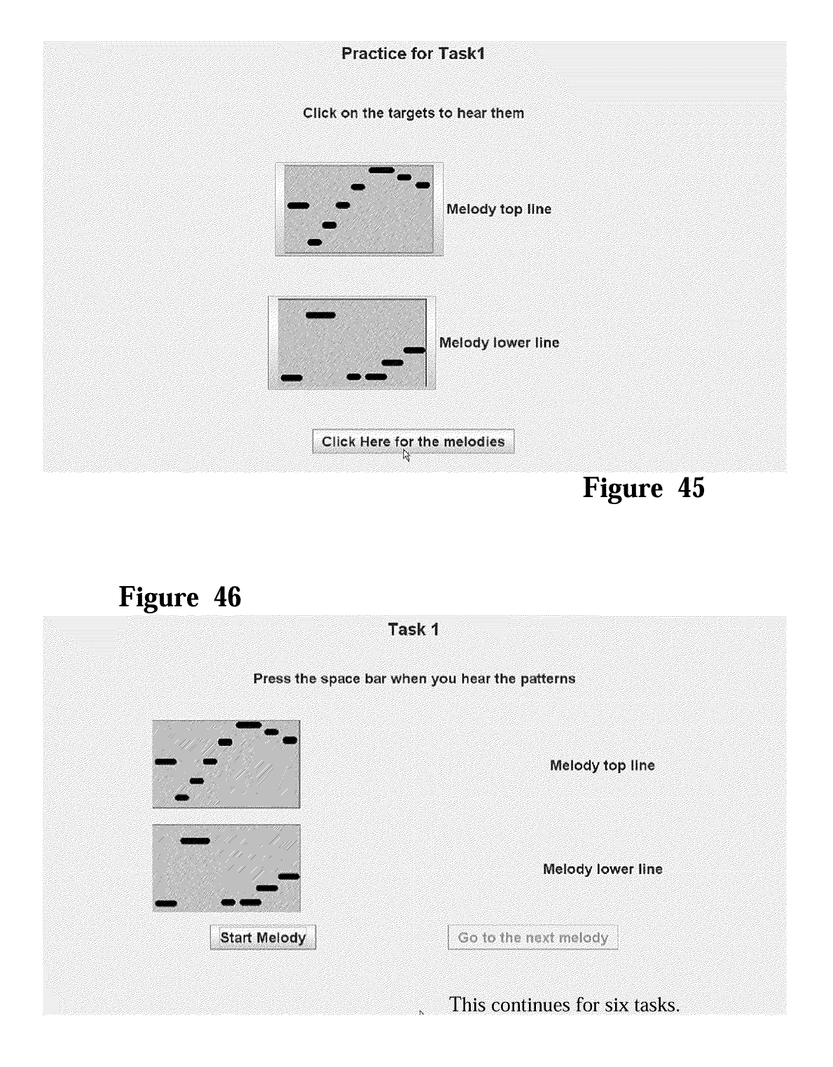

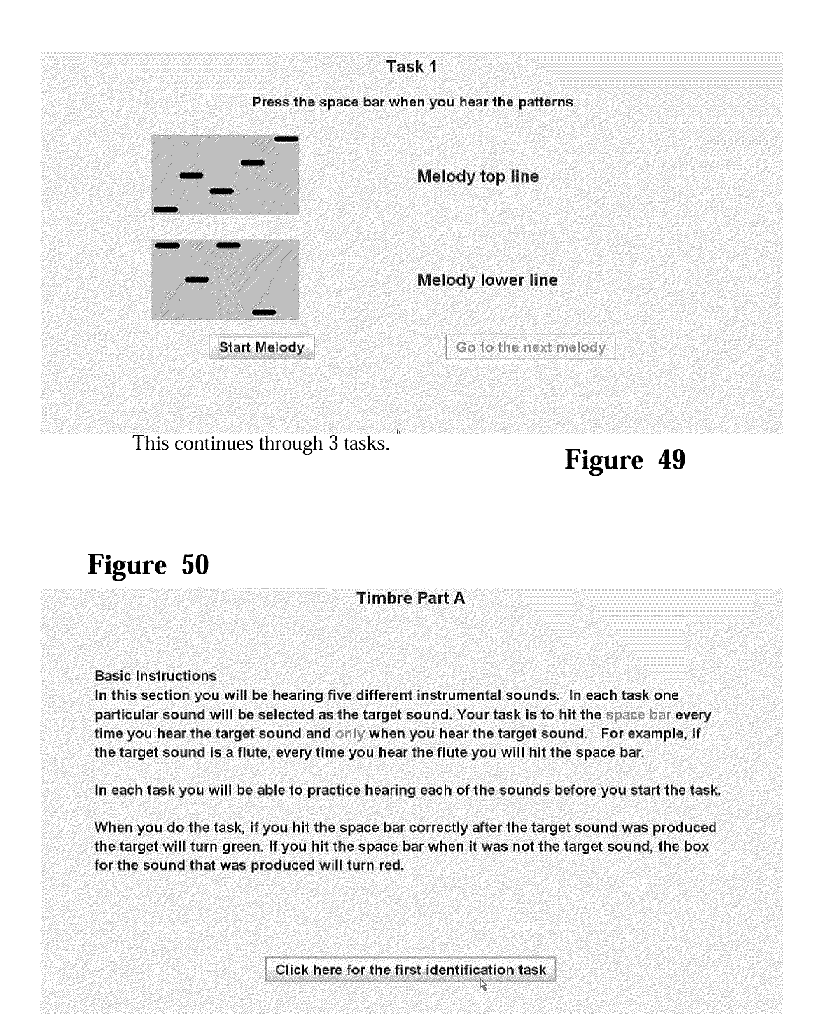

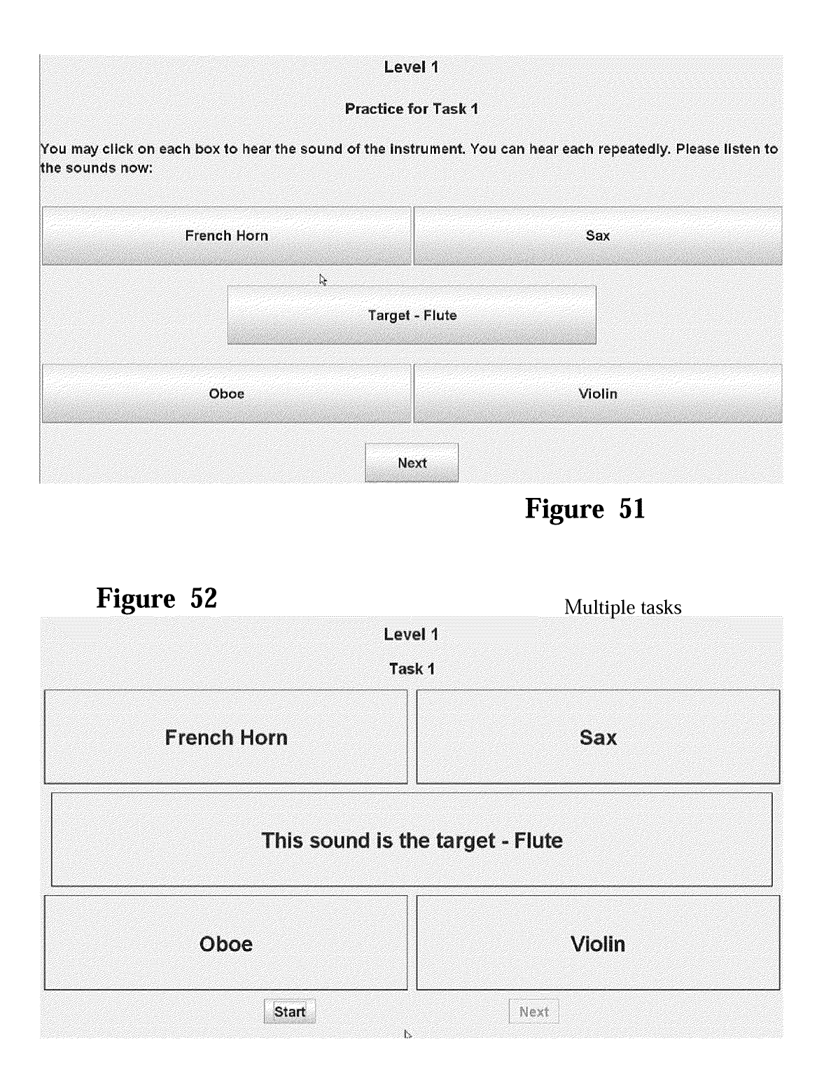

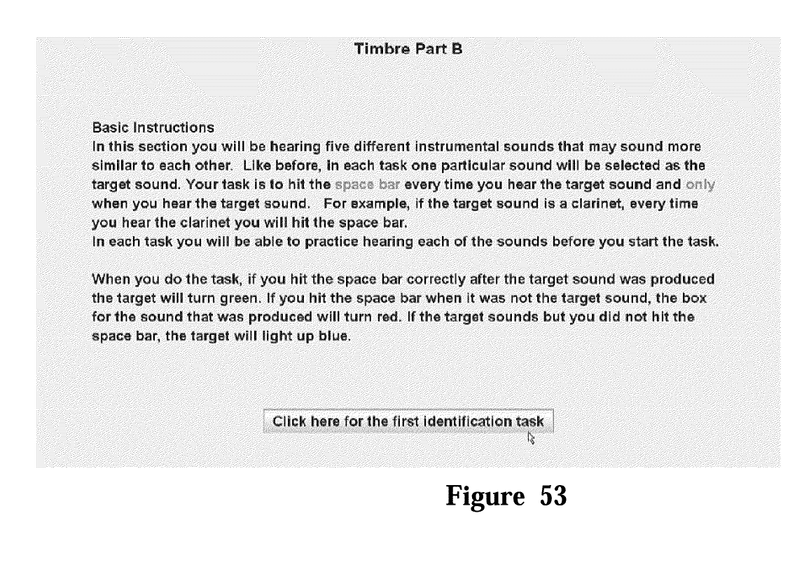

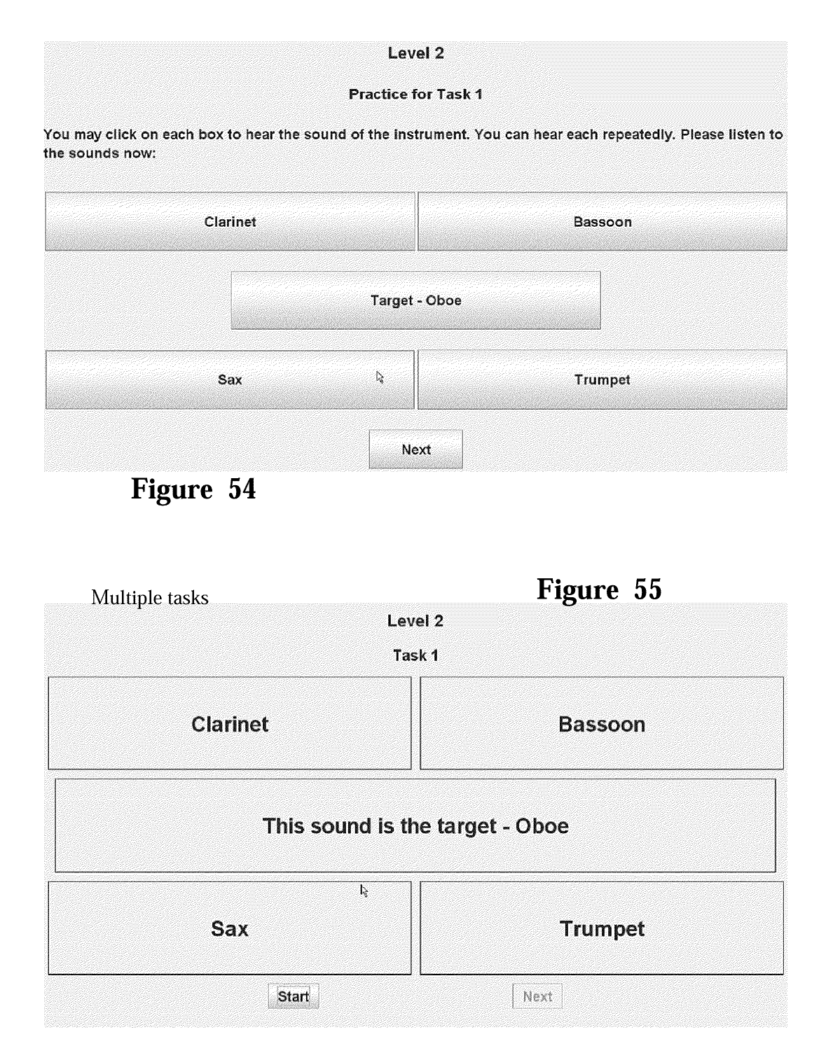

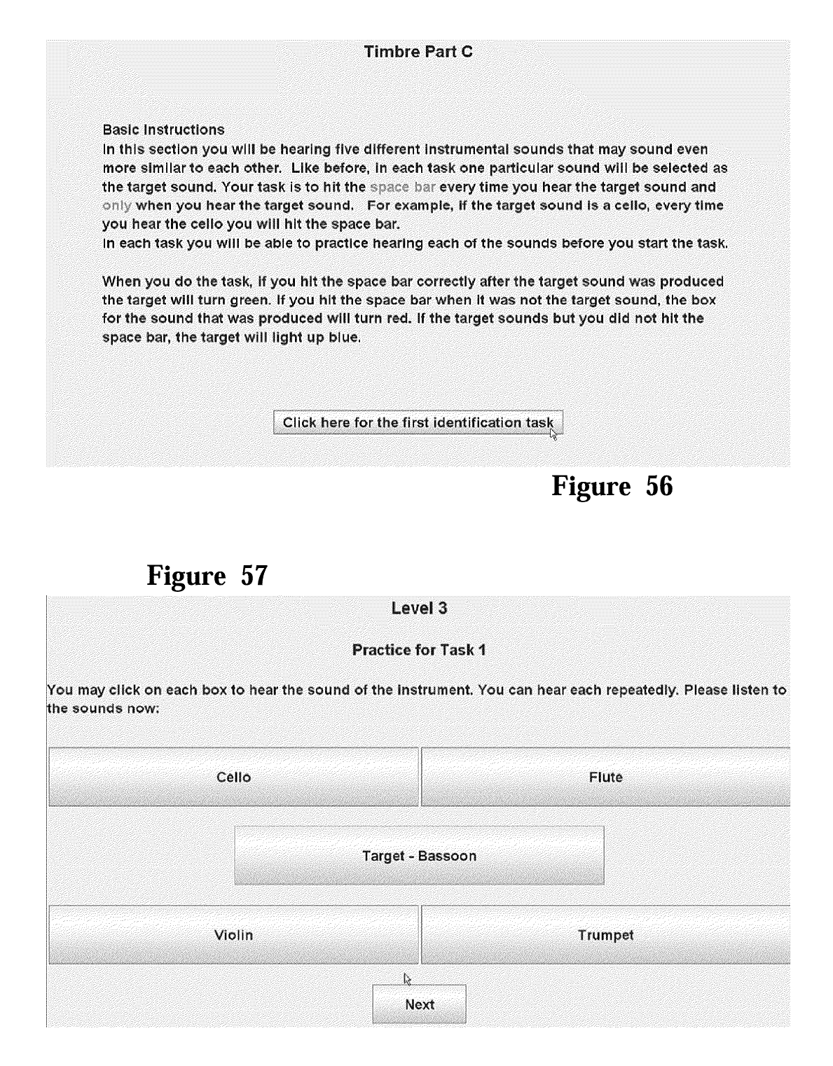

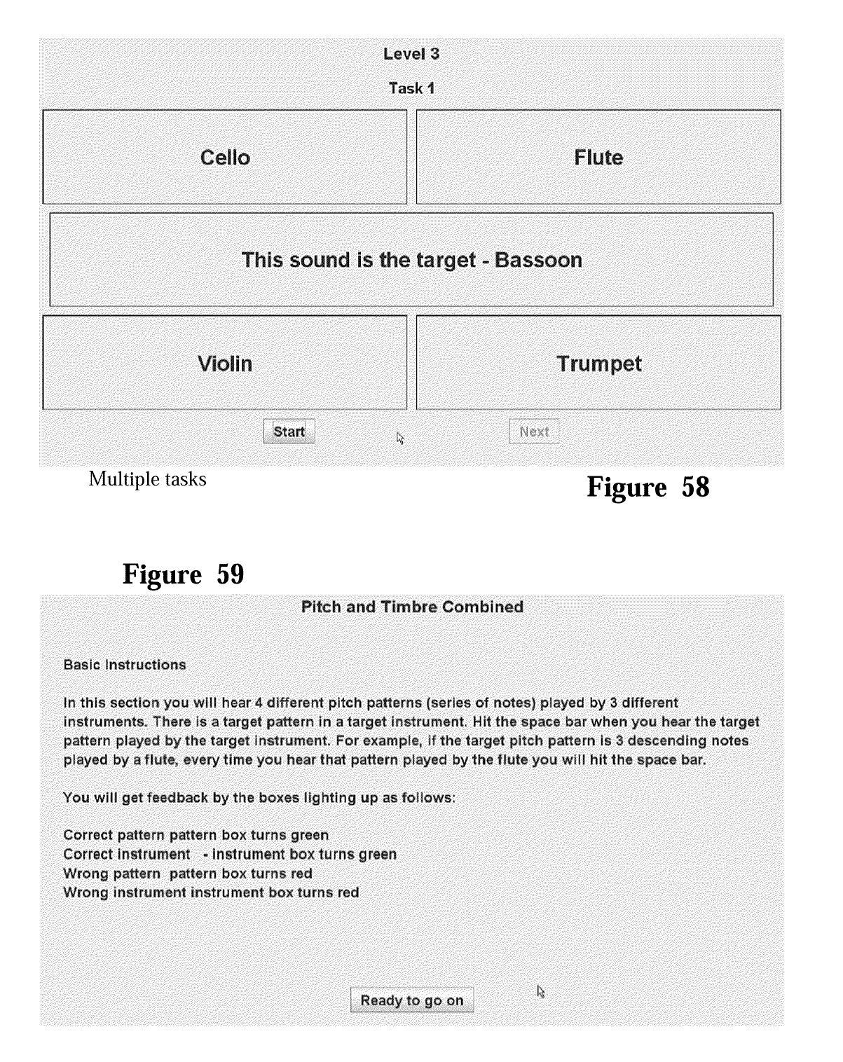

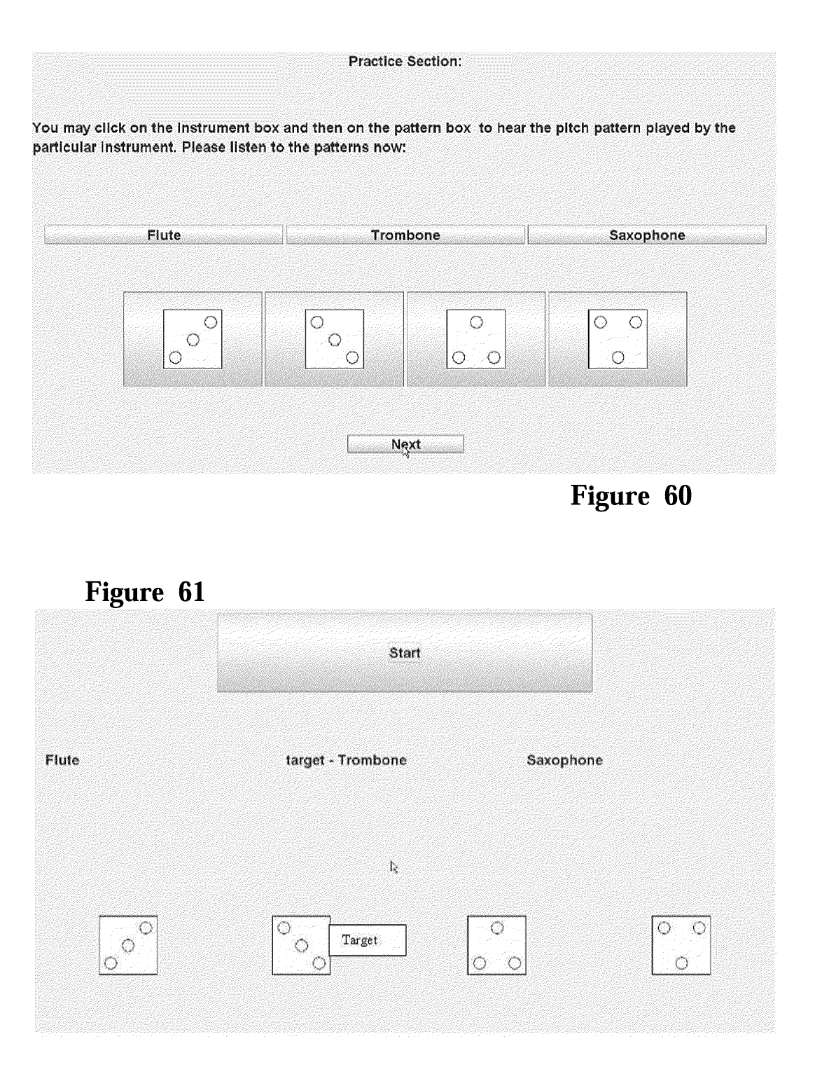

In the 3^{rd} Component the invention provides a Music Perception Assessment Tool (MPAT) preferably comprising a plurality of progressive levels in both diagnostic and rehabilitation modes as shown in FIGS. 20 to 61. At each pitch pattern level in the MPAT the invention visually presents 5 boxes to the subject for reaction.

At Level A of MPAT the invention includes sustained attention presentations and subject responses.

Level 1:

At the first pitch pattern level, the invention visually and audiologically presents the subject with a different 3 note musical pitch pattern graphically portrayed preferably

(1) ascending,

(2) descending,

(3) same pitch,

(4) low-high-low,

(5) high-low-high.

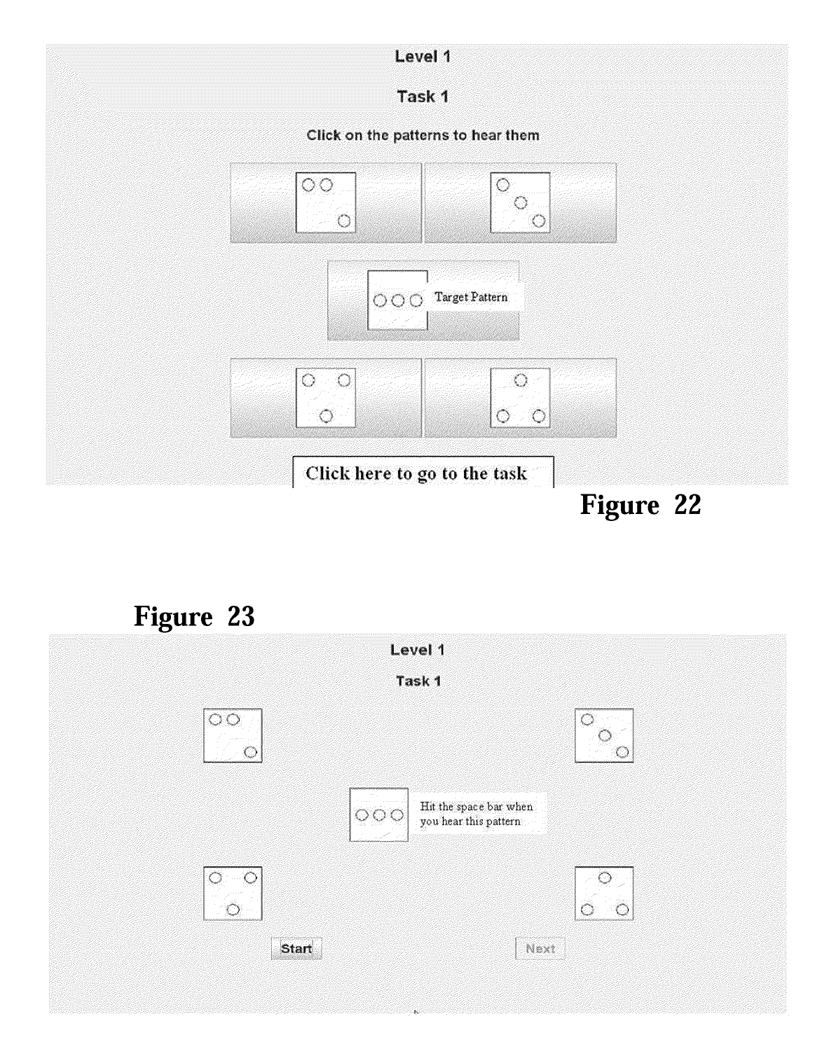

The range of pitch difference within each pattern as in FIGS. 22 to 27 is an interval of a major sixth. No rhythmic variation is used. The five boxes are presented on screen. The subject may pre-audition and/or practice each of them by selection and playback of the selected musical sound. The assessment tool is then activated and one pattern is randomly selected as the target. A visual identifier associated with this target (icon) moves to the middle screen position. The musical sounds are presented pattern after pattern—but with breaks between them that allow time for subject differentiation. When the target is heard the subject is required respond, preferably by pressing a switch immediately after. When the response is correct the target icon lights green. When the response is not the target sound, the target icon associated with the sound actually presented lights red.

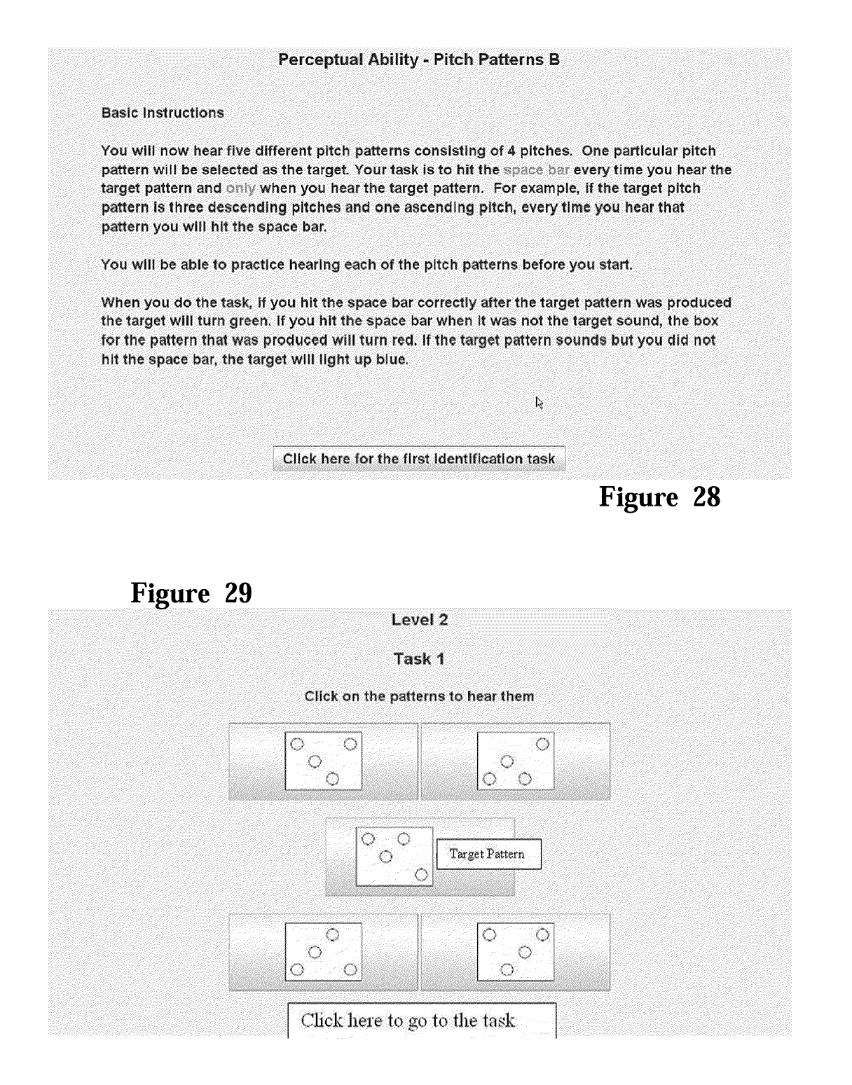

At Level 2: At the 2^{nd} pitch pattern level the invention visually and audiologically presents the subject with 4 note patterns that include 2 musical directions for reaction as in FIGS. 28 to 30. For example: high-medium-low-high, or low-medium-low-high, etc.

At Level 3: At the 3^{rd} pitch pattern level, the invention visually and audiologically presents the subject as in Level 1 but with narrower spaces between the musical tones as in FIGS. 31 to 33.

At Level 4: At the 4^{th} pitch pattern level, the invention visually and audiologically presents the subject as in Level 2 but with narrower pitch gaps for reaction.

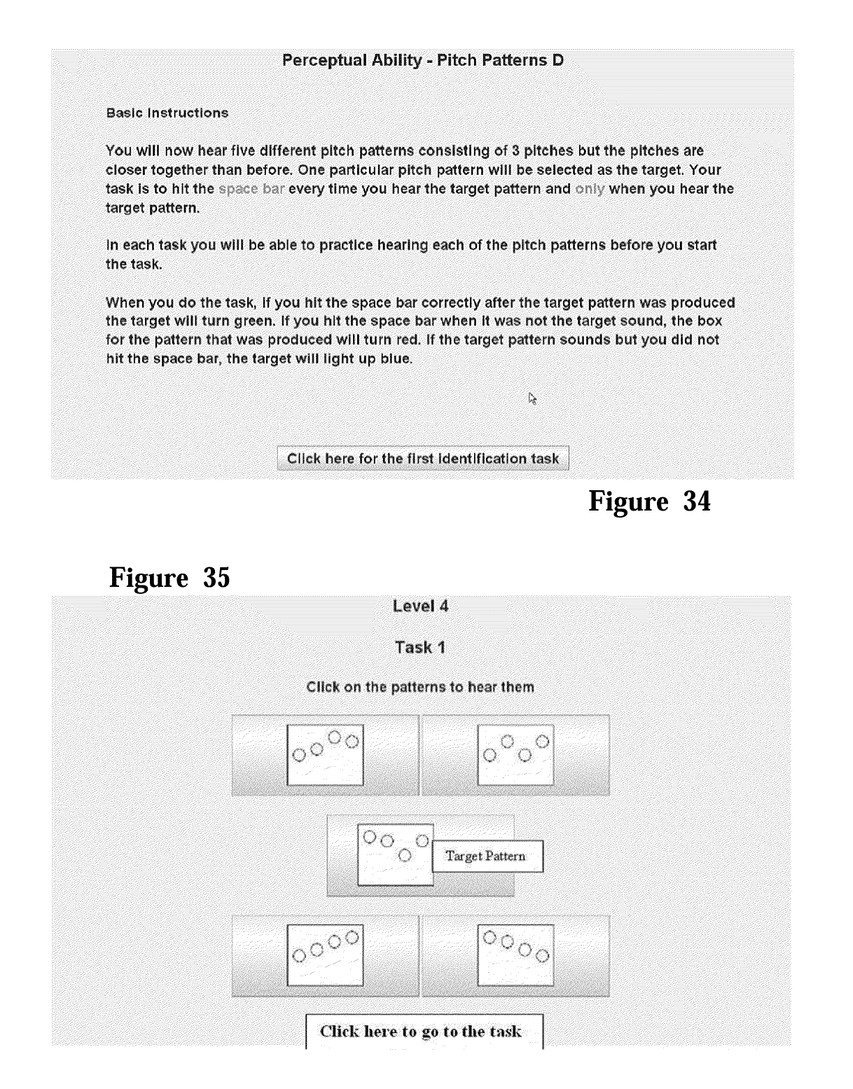

At a further Level, 5: At the 5^{th} pitch pattern level as in FIGS. 34 to 36, the invention visually and audiologically presents the subject with 3-note pitch patterns with 1 and 2 semi-tone gaps between pitches.

At a still further Level, 6: At the 4^{th} pitch pattern level, the invention visually and audiologically presents the subject with 3-note intervals presented with less rhythmic gap between patterns.

At all of the first 6 levels above in the diagnostic mode, 3 targets per tasks are presented per level and 3 tasks per level. When used in the training and rehabilitation mode, 4 targets per task are presented per level and a 75% mastery level is required before the invention moves the subject to the next task. In training and rehabilitation mode the invention generates the targets randomly until mastery is reached. Preferably, the mastery level is calculated by subtracting incorrect hits from correct.

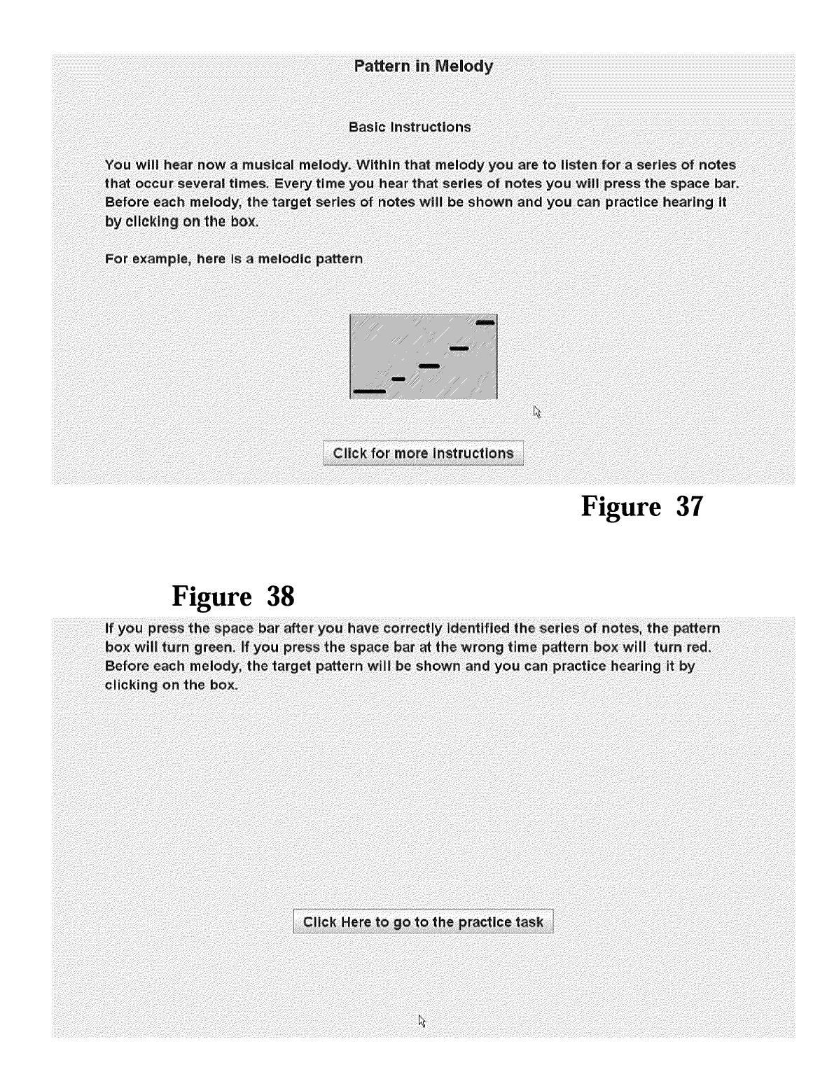

At level 7: The invention presents Musical Sound Patterns in Single-line Melodies directed at the Selective Attention capacity of the subject as in FIGS. 37 to 42.

At this level the invention plays a selected musical tune. A significant identifiable pattern segment from the melody is presented in an icon box as the target. The subject may listen to this target pattern repeatedly. When the subject is ready the melody is then played in a continuous form and the subject is expected to selectively respond every time the target pattern appears in the melody. Visual feedback is given by highlighting the target in green when the response is correct, or highlighting in red if it is incorrect.

The pattern and melody sections preferably are used both in diagnostics and in a mastery training manner. In training mode, if a 100% correct response is not received for a melody, the program goes into practice mode and the subject can hear the target pattern again, can hear the melody in a series of patterns, and can then go back to the task. When the correct response is received on each melody, the subject is advanced to the next level of difficulty.

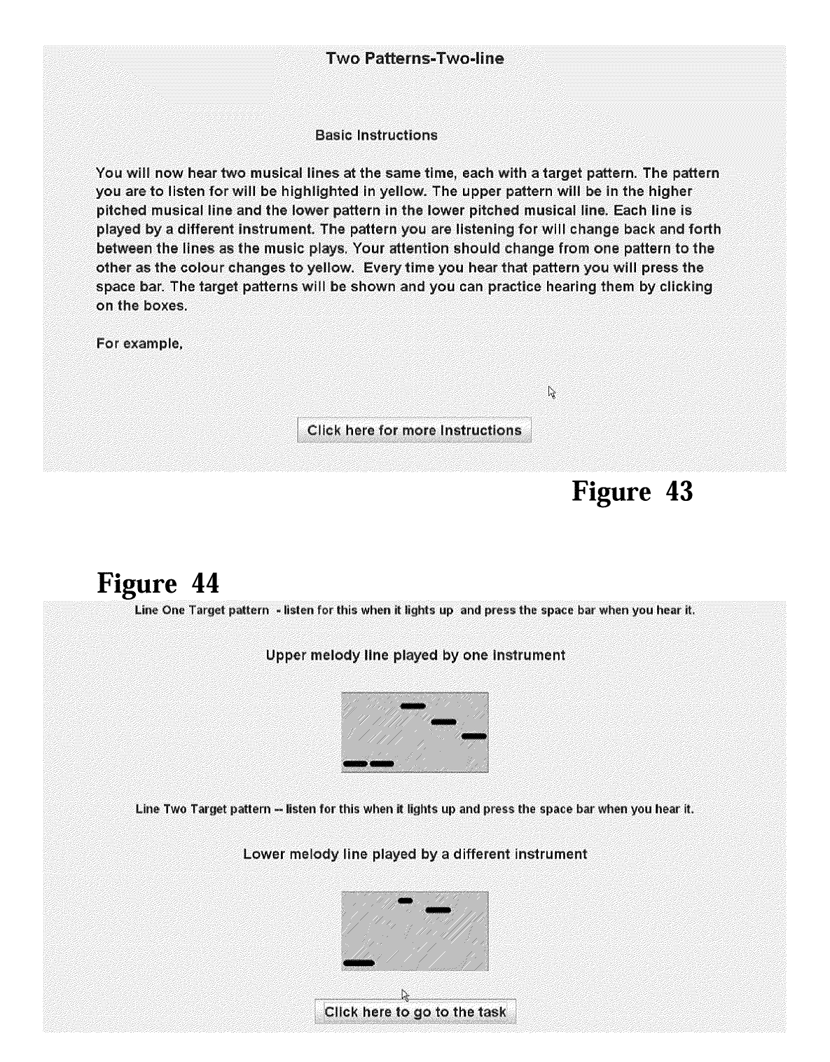

At level 8: The invention presents Musical Sound Patterns in Two-line Melodies directed at the Alternating Attention capacity of the subject as in FIGS. 43 to 46.

Two lines of music separated by an octave and featuring widely different timbres are presented simultaneously. They are represented visually by an upper line identifier and a lower line. A box appears over each line, each with a distinct identifiable pattern outline identifier (icon). The subject can select to hear the pattern. When the test is run, the invention shifts attention from one line to the other by lighting it up. The task is to attend to the line that lights up and to select when the target pattern is heard. The lines play at the same time but attention and scoring shifts from line to line according to the light.

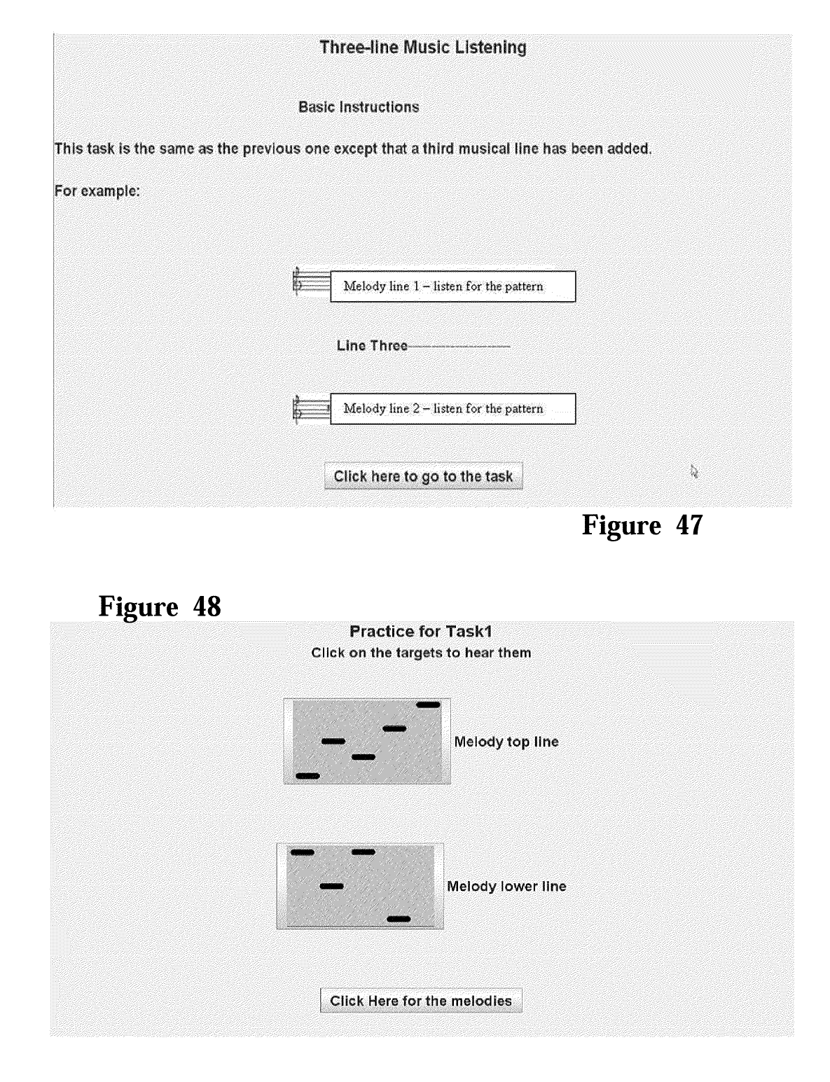

At level 9: The invention presents Musical Sound Patterns in Three-line Music Melodies also directed at the Alternating Attention capacity of the subject as in Level 8 as shown in FIGS. 47 to 49. The 3^{rd} line of music is, preferably, a music line between the two adding perceptual complexity (e.g., the targets are in soprano and bass).

At a further level, 10: The invention presents Musical Sound Patterns in Two-line Melodies directed at the Divided Attention capacity of the subject as shown for level 9.

Two lines of music are presented simultaneously, each separated by an octave and featuring widely different timbres. They are represented by an upper line identifier and a lower line. A box appears over each line, each with a distinct identifiable pattern outline identifier (icon). The subject selects to hear the pattern. When the invention is directed to proceed, both lines of music are presented simultaneously. The subject is required to attend to both lines and to select when the target pattern is heard in each line. No visual cuing to shift attention is provided.

At a further level, 11: The invention presents Musical Sound Patterns in Three-line Melodies directed at the Divided Attention capacity of the subject. The patterns are presented as in Level 10 but with a third line of music. More These advanced tasks add a third line as a complexity distractor. Preferably the targets are in soprano and bass but with a third line between them to add perceptual complexity.

In the 4^{th} Component the invention provides a Timbre Perception Ability Retraining Tool (TPART) including (1) identifying a target from a “line-up” of similar patterns; (2) visual feedback for response correctness, and (3) a stepwise increasing perceptual difficulty based on timbre theory, preferably comprising a plurality of progressive levels in both diagnostic and rehabilitation modes with 3 levels.

At Level A: At level A the invention presents 5 orchestral timbres that are at maximum timbral differentiation. Instruments associated are visually identified in a verbal label box (icon). Sounds can be heard by selecting each identifier before beginning the test. When start is selected, one of the timbres is randomly selected and the icon appears. The timbre of this instrument becomes the target. As the test runs, musical sounds are randomly played for a duration of 2 seconds. If the target timbre is played the subject is required to respond during or immediately after the musical sound. When the response is correct the target icon lights green. If the space bar is pressed when the response is not the correct target sound, the associated icon lights red. Timbral differentiation is based on Grey (1975, cited in Loy, 2006). Grey used a three dimensional hierarchical clustering analysis of subjects' timbre judgments to create a three dimensional mapping of timbre differences. The first dimension differentiating timbre in standard orchestral instruments is spectral energy distribution—many vs. fewer harmonics and how these are clustered or spread. The second dimension is the temporal energy distribution—how the harmonic partials (e.g., low vs. high harmonics) enter and exit the sound during attack and decay. This dimension accounts for most of the family of instrument timbre clustering. The third dimension also looks at temporal overtone patterns but particularly at high vs. low frequency and the amplitude at specific points during the sound.

At Level B: At level B the invention presents 5 orchestral timbres that are less clearly differentiated timbres than at level A.

At Level C: At level C the invention presents combined pitch patterns and timbre as in Level A as shown in FIGS. 59-61. The target pattern is set to be a particular note sequence and a particular timbre. When the subject hears combination a selection is made. For example, the target could be high-medium-low played by a flute. In the presentation sequence the pattern of high-medium-low might be presented played on an saxophone or other instrument. The screen presents two sets of boxes—the timbre choices and the pitch choices. The icons light up accordingly when the correct selection is made.

While particular embodiments of the invention have been described, it will be understood that the invention is not limited thereto, and that many obvious modifications and variations can be made, and that such modifications and variations are intended to fall within the scope of the appended invention.

What I claim is:

1. Response to sound measurement, diagnostic, retraining apparatus adapted for use with cochlear implants by recipients including:
(a) computer driven audio and visual stimulus presentation apparatus,
(b) selected and stored musical scores,
(c) recipient response apparatus,
(d) means to repeat said stimulation, selection and recipient responses,
(e) data collection and processing means, and
(f) means to access the cochlear implant including:
i. an optional Sound Enjoyment Assessment Tool,
ii. assessment retraining tools adapted to perform at least one of an assessment and retraining including at least one of:
A. a Perceptual Discrimination Assessment Tool including at least one of:
 a pitch discrimination assessment tool, and
 a timbre discrimination assessment tool,
B. a Music Perception Ability Tool, and
C. a Timbre Perception Ability Tool.

2. Response to sound measurement, diagnostic, retraining apparatus as claimed in claim 1 comprising multilevel recipient stimulus including means to provide increasing requirements for recipient attention and complexity.

3. Response to sound measurement, diagnostic, retraining apparatus as claimed in claim 2 wherein said recipient stimulus is adapted to provide a graded advancing complexity of musical sounds in direct relation to recipient attention demand levels.

4. Response to sound measurement, diagnostic, retraining apparatus as claimed in claim 3 wherein said recipient stimulus is adapted to provide recipient musical contour perception.

5. Response to sound measurement, diagnostic, retraining apparatus as claimed in claim 4 wherein said musical contour perception includes at least one of rising or falling patterns.

6. Response to sound measurement, diagnostic, retraining apparatus as claimed in claim 5 wherein said patterns initially provide an initial substantial frequency difference and a trend towards narrower frequency differences.

7. Response to sound measurement, diagnostic, retraining apparatus as claimed in claim 1 wherein said stimulus presentation means includes means to attenuate ambient sounds adapted to isolate said stimulus.

8. Response to sound measurement, diagnostic, retraining apparatus as claimed in claim 7 wherein said attenuation means includes a direct connection between said stimulus means and said implant.

9. Response to sound measurement, diagnostic, retraining apparatus as claimed in claim 7 wherein said visual stimulus acts in conjunction with said audio stimulus to prompt the recipient to select responses from a set of images.

10. Response to sound measurement, diagnostic, retraining apparatus as claimed in claim 9 wherein said attenuation means includes a direct connection between said stimulus means and said implant.

11. A response to sound measurement, diagnostic, retraining method adapted for use with cochlear implants by recipients including the steps of:
(a) computer driven audio and visual stimulus presentation,
(b) selection and storage of musical scores,
(c) obtaining recipient responses,
(d) repeating said stimulation, selection and recipient responses,
(e) data collection and processing, and
(f) accessing the cochlear implant 12. including:
i. optionally assessing Sound Enjoyment,
ii. assessing retraining adapted to perform at least one of an assessment and retraining including at least one of:
A. Perceptual Discrimination (PDAT) including at least one of:
 pitch discrimination (PA), and
 timbre discrimination (TA),
B. Music Perception Ability (MPAT).
C. Timbre Perception Ability (TPART).
